(19) United States
(12) Patent Application Publication
(10) Pub. No.: US 2024/0081843 A1
(43) Pub. Date: Mar. 14, 2024

(54) MINIMALLY INVASIVE METATARSAL REALIGNMENT

(71) Applicant: Treace Medical Concepts, Inc., Ponte Vedra, FL (US)

(72) Inventors: Adriaan Kuyler, Ponte Vedra, FL (US); Paul Dayton, Ankeny, IA (US); Mark Erik Easley, Durham, NC (US); William T. DeCarbo, Mars, PA (US); Daniel J. Hatch, Greeley, CO (US); Jody McAleer, Jefferson City, MO (US); Robert D. Santrock, Morgantown, WV (US); W. Bret Smith, Durango, CO (US); Sean F. Scanlan, Jacksonville, FL (US); Jason May, St. John's, FL (US); Michael Stedham, Jacksonville, FL (US)

(21) Appl. No.: 18/467,509

(22) Filed: Sep. 14, 2023

Related U.S. Application Data
(60) Provisional application No. 63/406,486, filed on Sep. 14, 2022.

(51) Int. Cl. A61B 17/17; A61B 17/02

(52) U.S. Cl. CPC A61B 17/1775; A61B 17/0218; A61B 2017/565

(57) ABSTRACT

Systems, techniques, and devices are described that may be used in a minimally invasive bone realignment procedure. In some examples, a method of performing a minimally invasive metatarsal correction procedure involves using a bone preparation guide having a guide surface with a length less than a diameter of a bone to be cut using the guide surface. The clinician can guide a bone preparation instrument along the guide surface and angle the bone preparation instrument beyond one or both ends of the guide surface to cut the end of the underlying bone beyond one or both of the ends.

### CROSS-REFERENCE

This application claims the benefit of U.S. Provisional Patent Application No. 63/406,486, filed Sep. 14, 2023, the entire contents of which are incorporated herein by reference.

### TECHNICAL FIELD

This disclosure relates generally to devices and techniques for repositioning bones and, more particularly, to devices and techniques for repositioning bones in the foot.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or attempting to realign the first metatarsal relative to the adjacent metatarsal. Surgical instruments that can facilitate efficient, accurate, and reproducible clinical results are useful for practitioners performing bone realignment techniques.

For patients, surgical intervention requires making one or more incisions through the patient's skin to access the underlying bones to perform a corrective procedure. A longer incision provides the surgeon with greater access to perform the procedure. However, a longer incision results in a longer scar for the patient after healing, which can be cosmetically undesirable. For this reason, the patient may prefer a shorter incision. This can be challenging for the surgeon because it limits access for performing the corrective procedure.

### SUMMARY

In general, this disclosure is directed to devices, systems, and techniques for performing a minimally invasive metatarsal realignment procedure that minimizes the length of one or more incision(s) utilized to access the bones for performing the procedure. As a result, a patient undergoing the procedure may have a shorter, less visible incision and resulting scar line, providing healing and/or cosmetic benefits as compared to a procedure performed using a longer incision.

In some implementations, a clinician surgically accesses a tarsometatarsal (“TMT”) joint defined between a metatarsal and an opposed cuneiform. The TMT joint may be the first TMT joint between the first metatarsal and medial cuneiform or a lesser TMT joint between a lesser metatarsal and opposed cuneiform. The clinician may utilize an incision guide placed over the skin of the patient to identify the correct location for cutting through the skin to surgically access the TMT joint. The incision guide may be used to mark the location for the incision and/or to guide a cutting instrument to make the incision through the skin of the patient.

In some applications, the clinician inserts a wire percutaneously (through the skin of the patient) into the TMT joint and then aligns the incision guide with a portion of the wire projecting out of the joint. Use of the incision guide can help ensure that the incision is small and precisely positioned relative to the TMT joint since the clinician may have limited surgical access through the small incision if incorrectly placed.

Independent of whether the clinician utilizes an incision guide, the clinician can surgically access the TMT joint and prepare the end face of the metatarsal and the end face of the opposed cuneiform. In some example, the clinician inserts a guide into the incision created through the skin of the patient. The guide can have one or more sidewalls defining an opening. The guide can be used to displace soft tissue at the incision location, e.g., by moving the soft tissue against the outer surface of the sidewalls, thereby providing surgical access through the opening in the guide while minimizing soft tissue interference. Additionally or alternatively, the clinician can utilize one or more retractors to retract skin and/or other soft tissue away from the incision location to help provide access to the underlying joint space.

Before and/or after preparing one or both end faces, the clinician may release soft tissue and/or boney protuberance(s) to help mobiles one or both bones at the TMT joint for realignment. For example, the clinician may insert a cutting instrument (e.g., osteotome, saw blade) dorsally into the TMT joint to release soft tissue within the joint and/or to cut one or more boney protuberances. Additionally or alternatively, the clinician may insert a cutting insert at the proximal lateral corner of the metatarsal to help mobilize the metatarsal for repositioning. In some such implementations, the clinician can insert a bi-planar tissue release instrument that includes two cutting bodies joined together, e.g., defining an angled corner (e.g., 90 degree corner) between each other. The bi-planar tissue release instrument can be inserted at the proximal lateral corner of the metatarsal, with one cutting body inserted into the TMT joint space and another cutting body inserted into a joint space between the metatarsal and an adjacent metatarsal. The bi-planar tissue release instrument can efficiently and effectively cut soft tissue through the small incision created in the skin of the patient.

An example surgical technique according to the disclosure can involve preparing the end face of the metatarsal and the end face of the opposed cuneiform. Example preparation steps may include reaming, cutting, rongeuring, curetting, burring, fenstrating and/or other similar techniques for exposing subchondral bone and/or establishing bleeding bone faces to promote fusion following rejoining of the metatarsal and the cuneiform. In some implementations, the clinician positions a bone preparation guide over the end of the metatarsal and/or the end of the cuneiform to be prepared. The bone preparation guide can have one or more guide surfaces, such as one or more slots, configured to guide a bone preparation instrument to prepare the end of the underlying bone.

In some examples, a bone preparation guide for a minimally invasive metatarsal procedure includes on more guide surfaces sized smaller than the size of one or more underlying bones to be prepared using the guide. For example, the bone preparation guide may include a slot through which a bone preparation instrument can be inserted to prepare an underlying bone (e.g., metatarsal, cuneiform). The slot may have a length shorter than the diameter of the underlying bone. Accordingly, when the bone preparation guide is positioned over the bone, the slot may cover a portion but not all of the bone to be prepared. This can facilitate use of a comparatively small bone preparation guide, allowing the clinician to make a smaller incision suitable for the comparatively small bone preparation guide than a longer incision that may be required if using a larger bone preparation guide.

To prepare the entire end face of the bone that the guide surface (e.g., slot) of the bone preparation guide partially but not fully covers, the clinician can manipulate a bone preparation instrument relative to the guide surface in multiple directions. For example, the clinician may translate the bone preparation instrument along the length of the guide surface to prepare that portion of the end face of the bone covered by the guide surface. When doing so, the bone preparation instrument may extend generally perpendicularly out of the bone preparation guide and be advanced linearly along the guide surface. Additionally or alternatively, the clinician may angle the bone preparation instrument relative to the length of the guide surface such that the axis of the bone preparation instrument is at an angle less than or greater than 90 degrees relative to the length of the guide surface. When the bone preparation instrument angled relative to the guide surface, the clinician may extend a distal portion of the bone preparation instrument under a sidewall of the bone preparation guide and/or over a sidewall of the bone preparation guide to cut one or more portions of the bone located beyond the end(s) of the guide surface (e.g., slot) of the bone preparation guide.

In some implementations, the bone preparation guide includes one or more sidewalls that include one or more sidewall cutouts. The sidewall cutout may extend partially but not fully along the height of the sidewall. In use, the clinician may angle the bone preparation insert across the sidewall and down into the sidewall cutout. The sidewall cutout can accommodate directing the bone preparation instrument at a sharper angle, e.g., to better access bone to be prepared beyond the sidewall, than if the sidewall does not include a cutout.

Before and/or after preparation of one or both end faces, the metatarsal can be realigned within one or more planes in three-dimensional space relative to the opposed cuneiform. The clinician may manipulate the position of the metatarsal by hand (e.g., optionally using a wire inserted into the bone) and/or with the aid of a bone positioner (also referred to as a bone positioning device). In some examples, the clinician engages a bone positioner with the metatarsal and a bone other than the metatarsal, such as the cuneiform and/or an adjacent metatarsal. The clinician can then user the bone positioner to apply a force to the metatarsal to move the metatarsal in one or more planes.

After moving the metatarsal relative to the opposed cuneiform and/or an adjacent metatarsal, the clinician can perform a variety of steps to complete the surgical procedure. In some examples, the clinician installs a compressor and utilizes the compressor to compress the prepared end face of the metatarsal together with the prepared end face of the cuneiform. Additionally or alternatively, the clinician may temporarily fixate a moved position of the metatarsal relative to the cuneiform. For example, the clinician can insert one or more fixation wires through the metatarsal into one or more adjacent bones (e.g., an adjacent metatarsal, across the TMT joint into the opposed cuneiform).

In either case, clinician may cause the metatarsal to fuse to the cuneiform. For example, the clinician may install one or more fixation devices across the TMT joint to fixate the position of the metatarsal relative to the cuneiform for subsequent healing and fusion. In some implementations, the clinician installs one more staples across the TMT joint. The one or more staples may apply a compressive force across the TMT joint after installation and may have a size effective to be installed through the comparatively small incision accessing the TMT joint. The clinician may additionally or alternatively install other fixation devices, such as plates, screws, rods, and/or the like.

In one example, a method of performing a minimally invasive metatarsal correction procedure is described. The method includes positioning at least one guide surface of a bone preparation guide over a metatarsal and/or a cuneiform of a patient to be prepared. The guide surface has a length extending from a first end to a second end, and the length of the guide surface is less than a diameter of the metatarsal and/or the cuneiform to be prepared. The method also involves guiding a bone preparation instrument along the guide surface to prepare an end of the metatarsal and/or an end of the cuneiform. According to the example, the technique may involve translating the bone preparing instrument along the length of the guide surface and angling the bone preparation instrument beyond one or both of the first end and the second end of the guide surface to prepare the end of the metatarsal and/or the end of the cuneiform beyond one or both of the first end and the second end of the at least one guide surface. The example technique can also involve moving the metatarsal relative to the cuneiform and fixating a moved position of the metatarsal.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIG. 4 is a flow diagram illustrating an example technique for performing a minimally invasive metatarsal realignment procedure.

FIGS. 5A and 5B are top and prospective side views, respectively, of one example incision guide that can be used to help set the size and/or location of an incision prior to making incision.

FIG. 6 is a dorsal view of an example foot showing a pin inserted percutaneously through the skin of the patient into the underlying TMT joint.

FIG. 7 is a dorsal view radiographic image of an example configuration of an incision guide showing an example positioning of the incision guide relative to the TMT joint.

FIG. 8 is a dorsal view of a foot also showing an example positioning of an incision guide relative to the TMT joint.

FIGS. 9A and 9B illustrate an example configuration of a pin and an incision guide, respectively, that can be used during a bone preparation and realignment procedure.

FIGS. 10A and 10B are top and perspective views, respectively, of an example retraction guide that may be inserted into an incision for retracting interfering soft tissue.

FIGS. 11A and 11B are top and perspective views, respectively, of another example retraction guide that may be inserted into an incision for retracting interfering soft tissue.

FIG. 12 illustrates an example configuration of a retraction guide inserted into an incision through the skin of a patient to help separate surrounding soft tissue underlying anatomical structure to be accessed by the clinician.

FIG. 13 illustrates one example cutting instrument that may be used during a surgical procedure.

FIG. 14 illustrates one example configuration of a retractor that may be utilized during a surgical procedure.

FIGS. 15A and 15B are perspective and top view illustrations, respectively, of an example retractor that can be used during a surgical procedure according to the disclosure.

FIGS. 16A and 16B are front and back perspective views, respectively, of an example biplanar tissue release instrument that can be used during a surgical procedure, such as a minimally invasive surgical procedure as described herein.

FIG. 17 is a perspective illustration of a foot showing an example configuration of a biplanar tissue release instrument inserted at a proximal-lateral corner of a metatarsal.

FIGS. 18A and 18B illustrate one example bone preparation guide that may be used as part of a minimally invasive procedure.

FIG. 19 is a dorsal view of an example foot illustrating an example configuration and positioning of the bone preparation guide of FIGS. 18A and 18B relative to one or more bones to be prepared using the guide.

FIG. 20 is a dorsal view radiographic image of an example foot showing an example configuration and positioning of the bone preparation guide of FIGS. 18A and 18B relative to one or more bones to be prepared using the guide.

FIG. 21 is a side view of an example configuration of the bone preparation guide of FIGS. 18A and 18B illustrating an example bone preparation instrument guided at an angle relative to a guide surface and extending beyond an end of the bone preparation guide.

FIGS. 22A-22D are different views of an example configuration of the bone preparation guide of FIGS. 18A and 18B illustrating example sidewall cutouts that may be utilized.

FIGS. 23A and 23B are sectional side views of an example bone preparation guide illustrating example sidewall cutout configurations that can be used on the bone preparation guide.

FIG. 24 is a perspective view of an example foot illustrating a first fixation pin inserted through a first fixation hole and percutaneously into an underlying metatarsal and a second fixation pin inserted through a second fixation hole and percutaneously into an underlying cuneiform.

FIGS. 25A and 25B are perspective illustrations of different configurations of an example bone preparation guide with raised of fixation holes.

FIGS. 26A and 26B are rear and side views, respectively, of an example bone preparation guide illustrating example tissue deflection features.

FIGS. 27A-27C are different views of an example hooked osteotome that may be inserted into the TMT joint and used to help extract a cut bone slice from the joint.

FIGS. 28A-28C are different views of an example bone slice grasping instrument that may be inserted into the TMT joint and used to extract a cut bone slice from the joint.

FIG. 29 shows a side perspective view of an example bone positioner that can be used to move a metatarsal relative to an adjacent bone.

FIG. 30 is a perspective view of an example foot illustrating an example compressing instrument that may be used to compress the prepared end faces of bones together.

### DETAILED DESCRIPTION

This disclosure generally relates to devices, systems, and techniques for performing a minimally invasive bone realignment procedure. In an exemplary application, the devices and techniques can be used during a surgical procedure performed on one or more bones, such as a bone alignment, osteotomy, fusion procedure, fracture repair, and/or other procedures where one or more bones are to be set in a desired position. Such a procedure can be performed, for example, on bones (e.g., adjacent bones separated by a joint or different portions of a single bone) in the foot or hand, where bones are relatively small compared to bones in other parts of the human anatomy. In one example, a procedure utilizing an embodiments of the disclosure can be performed to correct an alignment between a metatarsal (e.g. a first metatarsal) and a cuneiform (e.g., a medial cuneiform), such as a bunion correction. An example of such a procedure is a lapidus procedure. In another example, the procedure can be performed by modifying an alignment of a metatarsal (e.g. a first metatarsal). An example of such a procedure is a basilar metatarsal osteotomy procedure.

Preparation and fusion of two opposed bone portions, such as a metatarsal and cuneiform, may be performed according to the disclosure for a variety of clinical reasons and indications. Preparation and fusion of a metatarsal and cuneiform at the TMT joint may be performed to treat hallux valgus and/or other bone and/or joint conditions.

Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux. Consequently, the sesamoid bones may also be displaced, e.g., laterally relative to the first metatarsophalangeal joint, resulting in subluxation of the joints between the sesamoid bones and the head of the first metatarsal. This can increase the pressure between the medial sesamoid and the crista of the first metatarsal head.

While techniques and devices are generally described herein in connection with the first metatarsal and medial cuneiform of the foot, the techniques and devices may be used on other adjacent bones (e.g., separated from each other by a joint) and/or adjacent bone portions (e.g., portions of the same bone separated from each other by a fracture or osteotomy). In various examples, the devices, systems, and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof. The bones may be separated from each other by a tarsometatarsal (“TMT”) joint, a metatarsophalangeal (“MTP”) joint, or other joint. Accordingly, reference to a first metatarsal and medial cuneiform herein may be replaced with other bone pairs as described herein.

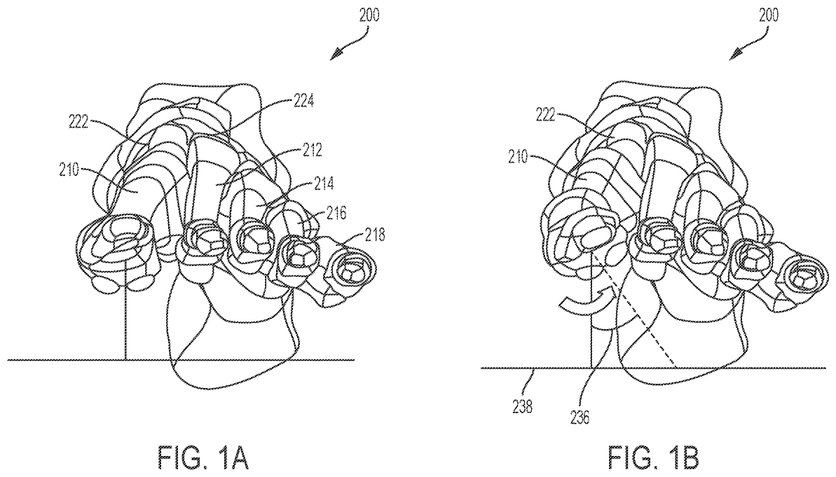

To further understand example techniques of the disclosure, the anatomy of the foot will first be described with respect to FIGS. 1-3 along with example misalignments that may occur and be corrected according to the present disclosure. A bone misalignment may be caused by hallux valgus (bunion), a natural growth deformity, and/or other condition.

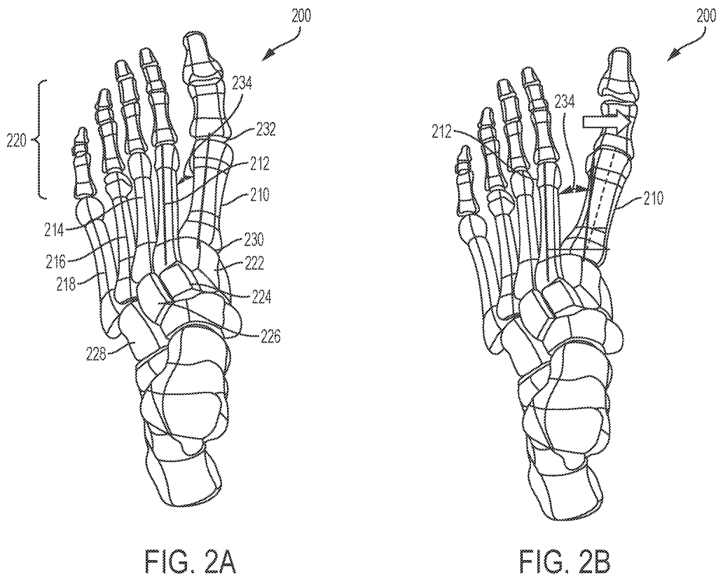

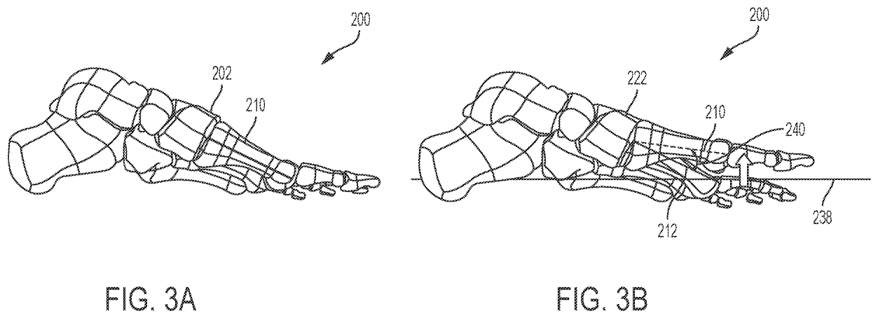

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The joint 232 between a metatarsal and respective proximal phalanx is referred to as a metatarsophalangeal (“MTP”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Surgical techniques and instruments according to the disclosure can be useful to correct a misalignment of one or more bones, such as the metatarsal and opposed cuneiform, and/or promote fusion of the metatarsal and cuneiform across the TMT joint. In some applications, the technique involves surgically accessing the TMT joint through a minimally-sized incision. The clinician may utilize an incision guide to identify the location and size of the incision to be made relative to the TMT joint prior to making the incision through the skin of the patient to surgically access one or both of the metatarsal and opposed cuneiform. After making the incision through the skin of the patient, the clinician may release soft tissue to mobilize the metatarsal for realignment and/or prepare an end of the metatarsal and an end of the cuneiform for fusion following realignment. The clinician may utilize one or more instruments configured for the minimally invasive procedure to retract the skin and/or other soft tissue around the small incision and to mobilize the metatarsal through the small incision.

The clinician can prepare the end face of the metatarsal and the opposed end face of the cuneiform with or without the aid of a bone preparation guide. In some implementations, the clinician utilizes a bone preparation guide having one or more guide surfaces configured to guide a bone preparation instrument relative to the underlying bone(s) to be prepared. Utilizing a bone preparation guide can provide more consistent and repeatable outcomes surgeon to surgeon and patient to patient, for consistent and efficacious clinical outcomes. In some configurations, the bone preparation guide positioned over the metatarsal and/or cuneiform has one or more guide surfaces that are size smaller than the bone to be prepared. In other words, the length of the guide surface may not extend across an entire width (e.g., diameter) of the metatarsal and/or cuneiform but may instead extend across only a portion of the width. Using a comparatively small bone preparation guide can allow the clinician to keep the size of the incision through the skin comparatively small as compared to using a larger bone preparation guide which may necessitate a larger incision.

When utilizing a bone preparation guide having a guide surface size smaller than the width of the underlying bone to be prepared, the clinician can manipulate a bone preparation instrument guided by the bone preparation guide to prepare the underlying bone beyond one or more boundaries defined by the guide surface. For example, the clinician may manipulate the bone preparation instrument to extend a distal portion of the instrument under a sidewall of the bone preparation guide. In use, the clinician may guide the bone preparation instrument along the guide surface to prepare a portion of the bone underlying the guide surface (e.g., by translating the bone preparation instrument linearly along the guide surface) and may angle the bone preparation instrument extend a distal portion of the instrument beyond an end of the guide surface. In this way, the clinician may prepare both the portion of the bone covered by the guide surface as well as the portion of the bone located beyond the end of the guide surface. In some configurations, the bone preparation guide includes one or more cut outs that allow the bone preparation instrument to be angled and/or manipulated beyond the end of the guide surface more efficiently than if the cutouts were not present.

Before and/or after preparing one or both of the end of the metatarsal in the end of the opposed cuneiform, the clinician can move the metatarsal relative to the cuneiform to realign the metatarsal in one or more planes. For example, the clinician may move the metatarsal relative to an adjacent metatarsal to close in intermetatarsal angle, rotate the metatarsal in a frontal plane to reposition the sesamoid bones under the metatarsal, and/or protonate or dorsiflex the metatarsal within the sagittal plane. The clinician may move the metatarsal in one or more planes directly with their hand (e.g., optionally using a pin, tenaculum, or other instrument) and/or may move the metatarsal in one or more planes with a bone positioning device operatively connected to the metatarsal. For example, the clinician may engage a bone positioning device with the metatarsal and also with a bone other than the metatarsal and then use the bone positioning device to apply a force moving the metatarsal in one or more planes.

With the metatarsal suitably repositioned, the clinician may temporarily and/or permanently fixate the moved position of the metatarsal relative to the cuneiform. For example, the clinician may insert a fixation pin through the metatarsal and into the cuneiform (e.g., across the TMT joint) to hold the position of the metatarsal for subsequent installation of one or more permanent fixation devices. In some implementations, the clinician compresses the prepared end face of the metatarsal together with the prepared end face of the cuneiform before installing one or more temporary and/or permanent fixation devices. For example, the clinician may install one or more staples, plates, screws, and/or pins to fixate the moved position of the metatarsal relative to the cuneiform, holding the moved position of the bones to allow the prepared end faces of the bones to subsequently fuse to each other.

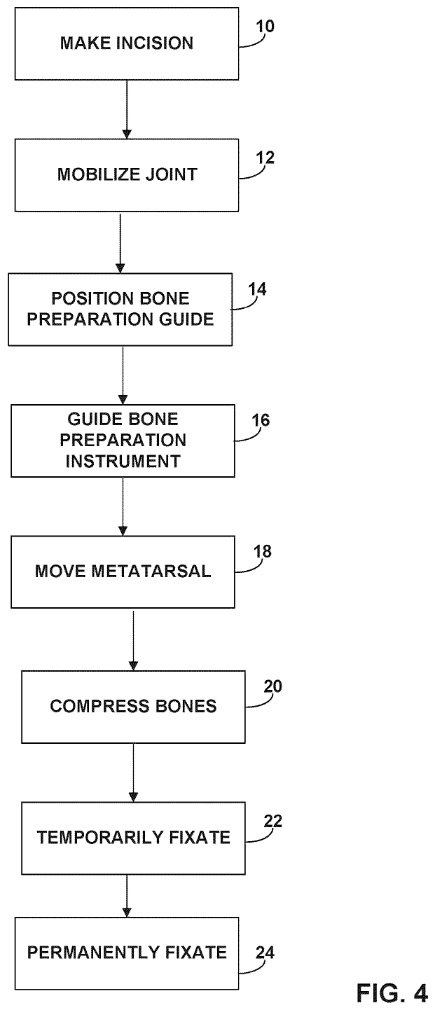

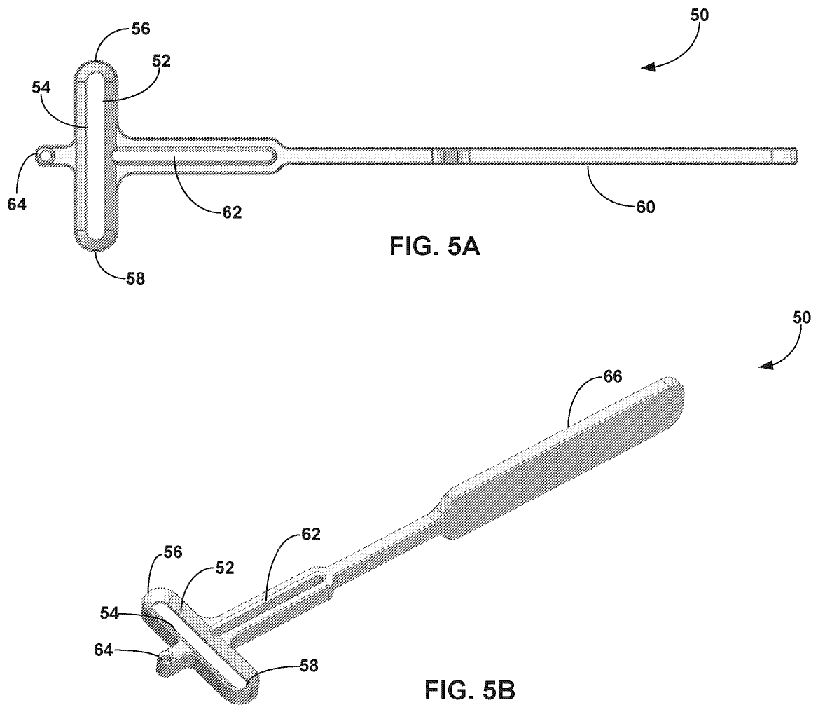

FIG. 4 is a flow diagram illustrating an example technique for performing a minimally invasive metatarsal realignment procedure. The example technique will be described with respect to first metatarsal 210 and medial cuneiform 222, although can be performed on other bones, as discussed above. For purposes of discussion, the technique of FIG. 4 will be discussed with respect to different example images illustrated in FIGS. 5-30.

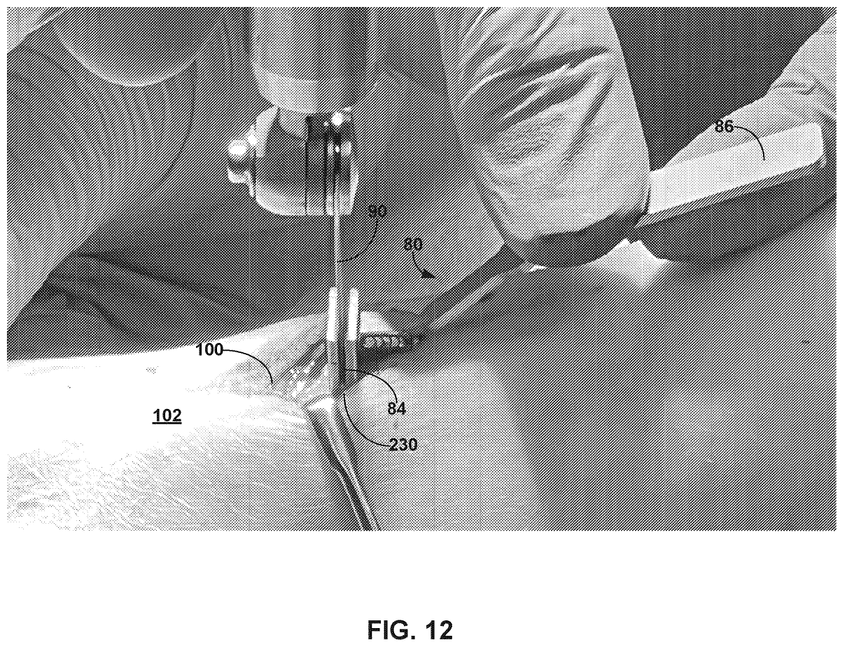

The example technique of FIG. 4 involves surgically accessing TMT joint 230 separating first metatarsal 210 from opposed medial cuneiform 222 (step 12 on FIG. 4). To surgically access the joint, the patient may be placed in a supine position on the operating room table and general anesthesia or monitored anesthesia care administered. Hemostasis can be obtained by applying thigh tourniquet or mid-calf tourniquet. An incision 100 (FIG. 12) can be made through the skin 102, such as on a dorsal side of the foot, a medial side of the foot, or on a dorsal-medial side of the foot. The incision may be made comparatively small to implement the minimally invasive procedure, reducing the size of the resulting scar line after the surgical procedure.

To help set the sizing and/or placement of the incision relative to TMT joint 230, the clinician may use an incision guide. FIGS. 5A and 5B (collectively referred to as “FIG. 5”) are top and prospective side views, respectively, of one example incision guide 50 that can be used to help set the size and/or location of the incision prior to making incision 100. Incision guide 50 can define one or more incision guide surfaces 52 for guiding an incision through the skin of the patient. In the illustrated example, incision guide 50 includes incision guide surface 52 and a facing incision guide surface 54 to define an incision guide slot between the two surfaces. Incision guide surface 52 (and the corresponding incision guide slot) has a length extending from a first end 56 to a second end 58. The length of the incision guide surface can be used to set the length of incision 100 to be made through the skin of the patient.

For example, in use, the clinician can position incision guide 50 over the skin of the patient at a location where incision 100 is to be made. The clinician can then make incision 100 by guiding a cutting instrument (e.g., scalpel) along the length of incision guide surface 52 and/or along the length of the incision guide slot between first and 56 and second end 58. Additionally or alternatively, the clinician can use incision guide 50 is a template to guide a marking source (e.g., a surgical marker pen) to indicate on the surface of the patient's skin where incision 100 is to be made. In these examples, the clinician may optionally remove incision guide 50 from the surface of the patient's skin and cut through the skin following the marked incision line. Incision guide 50 can help set the desired length of incision 100 to be made through skin 102 of the patient to effectuate the minimally invasive procedure, e.g., helping to prevent the clinician from making an overly long incision.

When performing a procedure through a comparatively small incision, the location of the incision may be precisely controlled to ensure sufficient surgical access at the location of the incision. This is because the clinician may not have the same flexibility to access one or more target anatomical areas through the comparatively small incision if the incision is improperly positioned relative to the underlying anatomy. In some implementations, the clinician may take a fluoroscopic (e.g. X-ray) image of at least a portion of foot 200 encompassing TMT joint 230 with incision guide 50 positioned over the skin of the patient and manipulate the position of incision guide surface 52 relative to the TMT joint using the fluoroscopic image. This can allow the clinician to position incision guide surface 52 at a target location relative to TMT joint 230 and first metatarsal 210 and medial cuneiform 222 defining the TMT joint. Once suitably positioned, the clinician can use the incision guide to make incision 100.

Additionally or alternatively, incision guide 50 may include or be configured to interface with a feature insertable into TMT joint 230. This can allow the location of TMT joint 230 to be identified with the insertable feature and incision guide 50 to be correspondingly positioned. In some examples, incision guide 50 is utilized as part of the system that includes a pin insertable into TMT joint 230. In use, the clinician can insert the pin percutaneously through the skin 102 of the patient into the joint and position incision guide 50 relative to the pin.

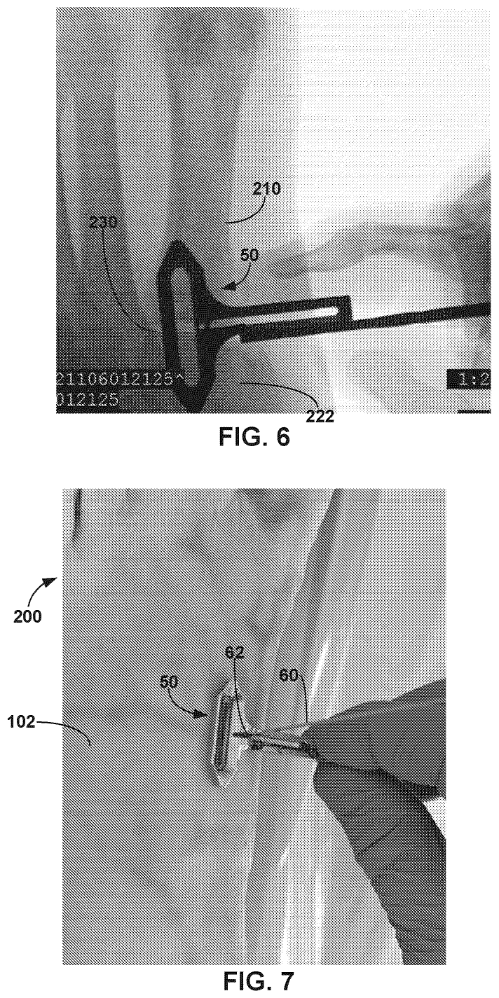

FIG. 6 is a dorsal view of an example foot 200 showing a pin 60 inserted percutaneously through the skin 102 of the patient into the underlying TMT joint 230. In some examples, the clinician may palpate foot 200 of the patient to identify TMT joint 230 and insert pin 60 percutaneously into the joint. Additionally or alternatively, the clinician may fluoroscopically view of foot 200 and insert pin 60 into an TMT joint 230 with the aid of fluoroscopic examination. In either case, pin 60 may have a length sufficient to position a distal portion of the pin at least partially, and in some examples fully, within TMT joint 230 while a proximal portion of the pin extends out of skin. Incision guide 50 can be operatively connected to pin 60, thereby aligning incision guide surface 52 with TMT joint 230 and/or metatarsal 210 and/or cuneiform 222 defining the joint. When used, pin 60 can have any cross-sectional shape (e.g., circular, square, triangular) and size. In some examples, pin 60 is comparatively small and may have a cross-sectional width (e.g., diameter) less than 2 mm, such as less than 1 mm.

With further reference to FIG. 5, incision guide 50 may include a pin receiving hole 62 that is configured to receive pin 60 inserted therein. For example, after inserting pin 60 into TMT joint 230, the clinician may connect incision guide 50 to pin 60 using pin receiving hole 62. In some examples, the clinician advances aligns pin receiving hole 62 of incision guide 50 with a proximal end of pin 60 and advances the incision guide down over the pin. In some implementations, pin receiving hole 62 has substantially a same size and shape as pin 60. In other implementations, pin receiving hole 62 is sized larger than pin 60 in at least one dimension to allow the relative movement between the incision guide and pin, after the incision guide is attached to the pin. For example, pin receiving hole 62 may be implemented as a slot that allows the incision guide to translate while pin 60 is received in the slot.

In use, the clinician may connect incision guide 50 to pin 60 by positioning the pin in the slot defined by the incision guide. The slot may be defined by sidewalls bounding distal-to-proximal movement of the incision guide. For example, the slot defined by the incision guide into which pin 60 is inserted may extend in a generally medial-to-lateral direction across TMT joint 230, e.g., generally parallel to the joint line. When so configured, the relative position of incision guide 50 in the distal-to-proximal direction along the length of metatarsal 210 and/or cuneiform 222 may be substantially set by the location of pin 60 in the sidewalls bounding the slot. However, the clinician may be able to move the incision guide, particularly the location of incision guide surface 52, e.g., in the medial-to-lateral direction in the transverse plane with pin 60 received in the slot. This can allow the clinician to set the specific location of the incision to be made along the width of TMT joint 230 after positioning the incision guide at a desired location in a distal-to-proximal direction.

In some examples, pin receiving hole 62 (e.g., the slot defined by the pin receiving hole) is substantially centered along the length of incision guide surface 52 between first end 56 and second end 58. When so configured, an incision guided by incision guide surface 52 can be substantially centered over TMT joint 230 (e.g., with approximately half of the incision over first metatarsal 210 and approximately half of the incision over medial cuneiform 222). In other examples, pin receiving hole 62 may be offset relative to a center of the length of incision guide surface 52. Incision guide 50 can include a handle 66 that a clinician can grasp to position and hold the incision guide during use and/or to manipulate the position of the incision guide relative to the anatomy of the patient.

Incision guide 50 may include one or more additional pin receiving holes 64 to receive one or more additional pins, e.g., inserted percutaneously through the one or more additional pin receiving holes into underlying bone. For example, incision guide 50 may include a pin receiving hole 64 on an opposite side of incision guide surface 52 from pin receiving hole 62 and/or adjacent first end 56 and/or second end 58 of the incision guide. Inserting additional pins into incision guide 50 may help stabilize the incision guide relative to the underlying skin and bones, e.g., when using the incision guide to guide a cutting instrument along incision guide surface 52.

When using incision guide 50, the incision guide can be placed at any suitable location about TMT joint 230, such as on a dorsal side of the joint, a medial side of the joint, or on a dorsal-medial side of the joint. In some implementations, incision guide surface 52 of incision guide 50 (and/or a widthwise center of a slot defined by the guide surface) is positioned over skin 102 of the patient on a dorsal side of TMT joint 230. Incision guide surface 52 (and/or the slot defined by the guide surface) may be aligned with a longitudinal centerline of metatarsal 210 and/or cuneiform 222 across TMT joint 230 on the dorsal side, or the incision guide surface (and/or the slot defined by the guide surface) may be displaced medially or laterally relative to the longitudinal centerline. Positioning incision guide surface 52 (and/or the slot defined by the guide surface) offset from the longitudinal centerline of the joint may be useful for preferentially accessing a medial or lateral side of the TMT join for subsequent steps of the procedure.

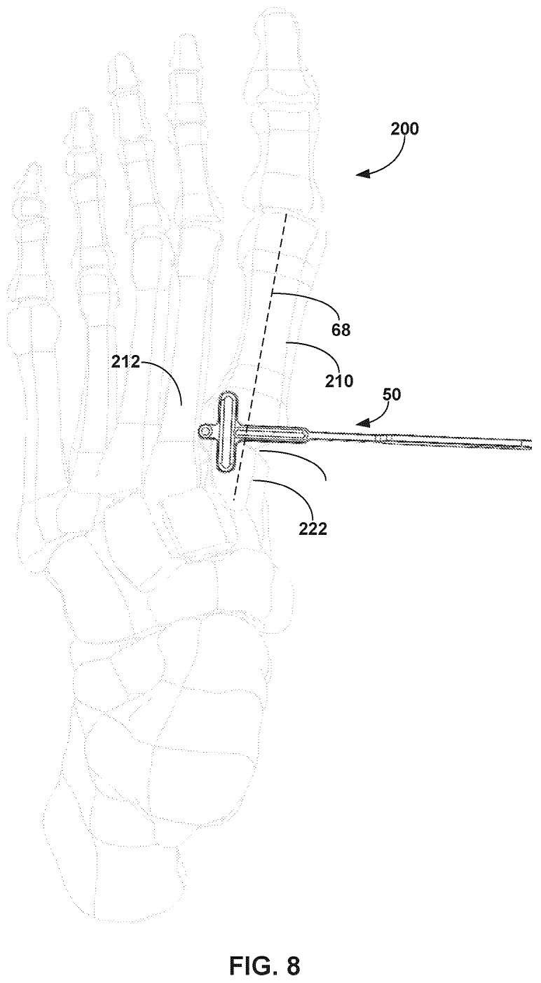

FIG. 7 is a dorsal view radiographic image of an example configuration of incision guide 50 showing an example positioning of the incision guide relative to TMT joint 230. FIG. 8 is a dorsal view of foot 200 also showing an example positioning of incision guide 50 relative to TMT joint 230. In the example of FIGS. 7 and 8, an incision slot defined by incision guide 50 is illustrated as being positioned lateral to a longitudinal axis 68 bisecting metatarsal 210 and cuneiform 222 across TMT joint 230 in the proximal-to-distal direction. Longitudinal axis 68 can divide TMT joint 230 into a medial half and a lateral half in the transverse plane. In some examples, incision guide surface 52 and/or a widthwise center of an incision slot defined by the guide surface may be positioned on the lateral half of TMT joint 230 (e.g., while being substantially centered in the distal-to-proximal direction), such as the lateral-most third of the joint, or lateral-most quarter of the joint. For example, incision guide surface 52 and/or a widthwise center of an incision slot defined by the guide surface may be positioned laterally at least 1 cm from longitudinal axis 68, such as at least 2 cm, at least 3 cm, or from 1 cm to 3 cm. In other configurations, incision guide surface 52 and/or a widthwise center of an incision slot defined by the guide surface may be positioned medially of longitudinal axis 68 any of the locations and dimensions described as being laterally displaced. In still other configurations, incision guide surface 52 and/or a widthwise center of an incision slot defined by the guide surface may be positioned substantially over longitudinal axis 68.

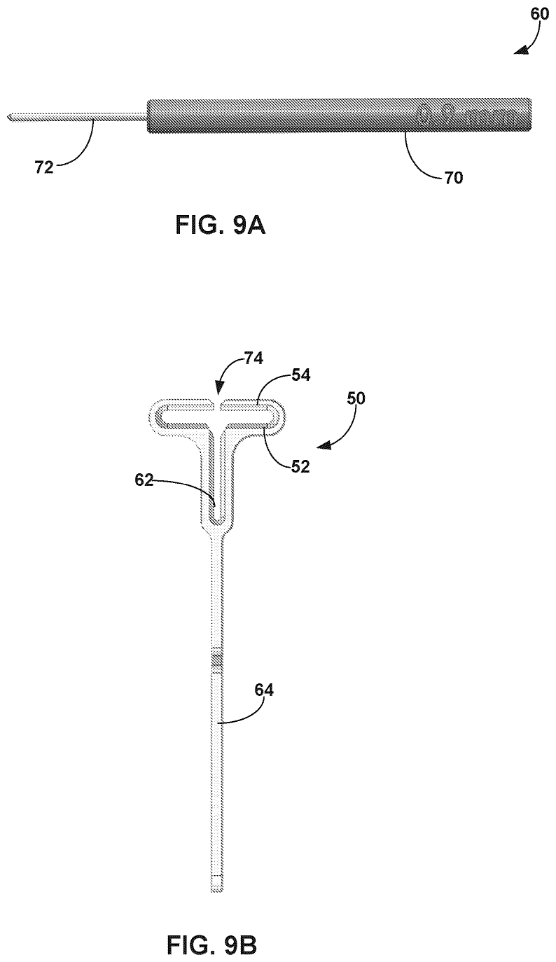

As noted, incision guide 50 and pin 60 can have a variety of different configurations. FIGS. 9A and 9B illustrate an example configuration of pin 60 and incision guide 50, respectively, that can be used during a bone preparation and realignment procedure. As shown in FIG. 9A, pin 60 may have a comparatively smaller distal portion 70 insertable percutaneously into TMT joint 230 and a comparatively larger proximal portion 72. Larger proximal portion 72 may have a cross-sectional width (e.g., diameter) at least 50% bigger than distal portion 70, such as at least 100% bigger. Larger proximal portion 72 may provide an enlarged region for a clinician to grasp when inserting distal portion 70 through skin 102 of the patient into TMT joint 230. Incision guide 50 can be installed over a section of distal portion 70 projecting out of the skin of the patient and/or a portion of larger proximal portion 72.

FIG. 9B illustrates an example configuration of incision guide 50 that includes a channel 74 through insertion guide surface 52 and facing insertion guide surface 54. Channel 74 is connected to pin receiving hole 62, which is shown as a slot extending parallel to handle 66 of the incision guide. Configuring incision guide 50 with channel 74 may be useful to connect the incision guide to pin 60 when the pagan is configured with a larger portion, e.g., graspable by the clinician. Incision guide 50 can be aligned with a portion of pin 60 extending out of the TMT joint 230 (e.g., a portion of smaller distal portion 70) by advancing channel 74 over the pin until the pin is positioned in pin receiving hole 62. Other configurations of an incision guide can be used without departing from the scope of the disclosure. For example, while incision guide 50 and pin 60 are described is to separate components that can be aligned with each other, in other examples, incision guide 50 may include an integrally formed pin or other alignment feature (e.g., extending from a bottom side of the incision guide) that is not separable from the incision guide.

Independent of whether the clinician uses an incision guide or the specific configuration of the incision guide, the clinician can cut through the skin of the patient to make an incision for accessing anatomy underlying the incision location. While the specific length of incision 100 (in the distal to proximal direction parallel to the long axis of first metatarsal 210) may vary, in some examples, the incision may have a length less than 6 cm, such as a length less than 5 cm, less than 4 cm, less than 3 cm, less than or equal to 2 cm, less than or equal to 1.5 cm, or less than or equal to 1 cm. In some instances, incision 100 has a length ranging from 1 cm to 4 cm, such as from 1.5 cm to 3 cm, from 1.5 cm to 2.5 cm, from 1.8 cm to 2.2 cm, or approximately 2 cm (e.g., ±5%). The length of the incision guide surface and/or incision guide slot defined by an incision guide to cut such incision may fall within any of the foregoing values.

With TMT joint 230 exposed through incision 100 in the skin 102 of the patient, the example technique of FIG. 4 can include mobilizing the TMT joint to facilitate subsequent repositioning of metatarsal 210 (step 14 on FIG. 4). For example, after creating surgical access to the joint and before moving the metatarsal to a realigned position, the clinician may cut soft tissue and/or obstructing bone to allow the metatarsal to move comparatively freely for realignment. For example, the clinician may insert a cutting instrument (e.g., saw blade, osteotome) into TMT joint 230 between a metatarsal 210 and cuneiform 222 and/or in the intermetatarsal joint space between metatarsal 210 and an adjacent metatarsal 212. The clinician may insert the cutting instrument into the target joint space(s) to cut soft tissue (e.g., muscles, tendons, ligaments, and/or facia) within the joint space. Such soft tissue may be connectively attached to the bone (e.g., metatarsal) intended to be realigned. Cutting the soft tissue can mobilize the bone for subsequent realignment. Additionally or alternatively, the clinician may excise (cut) obstructing bone, such as a dorsolateral flare of the metatarsal base, a plantar flare of the metatarsal base (sometimes referred to as a plantar condyle), part of an end of a metatarsal facing a cuneiform, and/or an osteophyte to help promote free rotation of the metatarsal by creating relatively flat surfaces with respect to a frontal plane.

When surgically accessing one or more joint spaces through a comparatively small incision, soft tissue (e.g., skin, tendons such as the extensor hallucis longus (EHL) tendon) may have a tendency to close over the incision space, interfering with surgical access to underlying tissue and/or bone. Accordingly, in some examples, the clinician may insert a retraction guide into the incision to help separate soft tissue from underlying anatomical structure. The retraction guide can offset potential interfering soft tissue, exposing underlying anatomical structure for surgical access by the clinician. A variety of different retraction guide designs can be used.

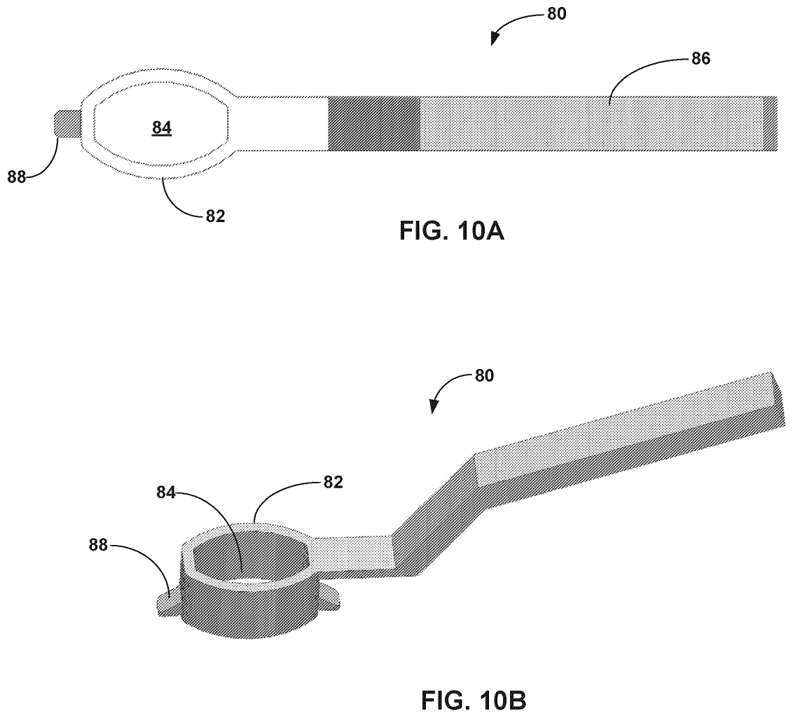

FIGS. 10A and 10B are top and perspective views, respectively, of an example retraction guide 80 that may be inserted into incision 100 for retracting interfering soft tissue. Retraction guide 80 can include at least one sidewall 82 defining a partially or fully enclosed opening 84 through which the clinician can surgically access underlying anatomical structure. Retraction guide 80 can be implemented in a variety of different sizes and shapes, an opening 84 can have any desired polygonal (e.g., square, rectangle, triangle) and/or arcuate shape (e.g., circle, oval). In use, the clinician can insert retraction guide 80 into incision 100, using sidewall 82 of the retraction guide to displace interfering soft tissue while providing surgical access through opening 84 of the retraction guide.

In the illustrated example, retraction guide 80 is shown as including a handle 86 that is graspable by the clinician to position and/or hold the retraction guide in incision 100 during use. The illustrated example also shows retraction guide 80 having at least one outwardly extending foot 88, which is illustrated as being implemented with a pair feet 88 extending in opposite directions, from the at least one sidewall 82. The one or more feet 88 may be positioned under the skin 102 of the patient when retraction guide 80 is placed in incision 100, helping to hold and retain the retraction guide in the incision location during subsequent use.

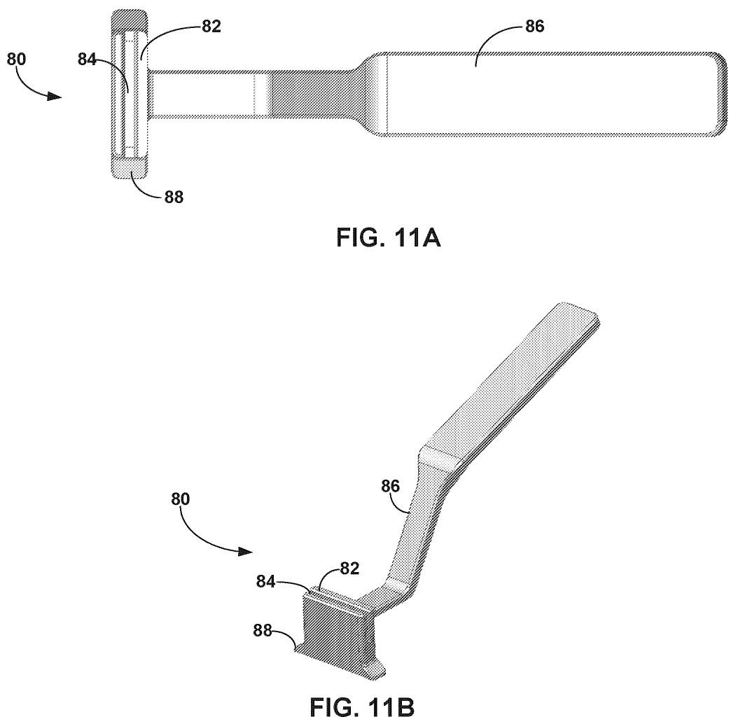

FIGS. 11A and 11B are top and perspective views, respectively, of another example retraction guide 80 that may be inserted into incision 100 for retracting interfering soft tissue. Like features in FIGS. 11A and 11B to those discussed above with respect to FIGS. 10A and 10B are designated by like reference numerals. Retraction guide 80 in the examples of FIGS. 11A and 11B is illustrated as defining a rectangular opening 84 sized to have a planar cutting instrument (e.g., osteotome, saw blade) inserted through the opening to cut underlying tissue and/or bone.

FIG. 12 illustrates an example configuration of retraction guide 80 inserted into an incision 100 through the skin 102 of a patient to help separate surrounding soft tissue underlying anatomical structure to be accessed by the clinician. In particular, in the illustrated example, the configuration of retraction guide 80 having a rectangular shaped opening (as illustrated in FIGS. 11A and 11B) as shown inserted into an incision 100 with the opening 84 of the retraction guide positioned over TMT joint 230. The feet 88 of retraction guide 80 are positioned under the skin 102 of the patient to help retain the retraction guide under the skin, and handle 86 is illustrated as extending along the longitudinal axis of the metatarsal. In the illustrated example, a saw blade 90 is inserted through the slot-shaped opening 84 of retraction guide 80 into TMT joint 230 to cut tissue and/or bone for mobilizing the metatarsal for subsequent repositioning.

The clinician may utilize a variety of different retraction guide designs (or may not even utilize a retraction guide). When utilizing a retraction guide, the opening 84 of the retraction guide may be positioned at any suitable location in the over any target anatomy to be surgically accessed by the clinician. Further, when releasing soft tissue and/or bone to help mobilize the metatarsal for realignment, the clinician may utilize any desired cutting instrument. The cutting instrument may be a reciprocating or oscillating instrument connected to a powered hand unit or may be directly grasp by the clinician with the cutting force delivered by movement of the clinician's hand.

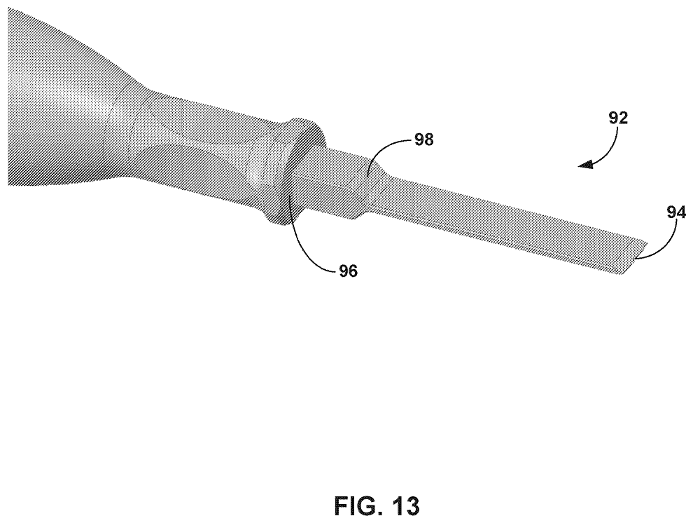

FIG. 13 illustrates one example cutting instrument 92 that may be used during a surgical procedure. For example, cutting instrument 92 may be used to release TMT joint 230 by cutting soft tissue and/or one or more bony projections into the joint space to help mobilize the metatarsal for realignment. Cutting instrument 92 is illustrated as having a length extending from a first end 94 to a second end 96. The first end 94 of cutting instrument 92 may be tapered in one or both planes across the thickness of the cutting instrument to provide a sharpened leading edge. In the illustrated arrangement, cutting instrument 92 includes a depth stop 98 positioned proximally of a distal end of the cutting instrument.

In some examples, the length of cutting instrument 92 may be sized relative to the anticipated anatomy of the patient undergoing the procedure and/or retraction guide 80 to be used with the procedure. For example, cutting instrument 92 may have a length from first end 94 to depth stop 98 sized to allow the cutting instrument to be advanced through retraction guide 80 and into underlying anatomy (e.g., TMT joint 230) to a desired depth. Cutting instrument 92 may have a substantially constant thickness over this length of the instrument. Depth stop 98 may be an outwardly extending feature and/or a region of enlarged cross-sectional thickness that is configured to contact a top surface of sidewall 82 of retraction guide 80. For example, the clinician may plunge cutting instrument 92 through opening 84 of retraction guide 80 in into underlying anatomy until depth stop 98 contacts sidewall 82 of the depth stop.

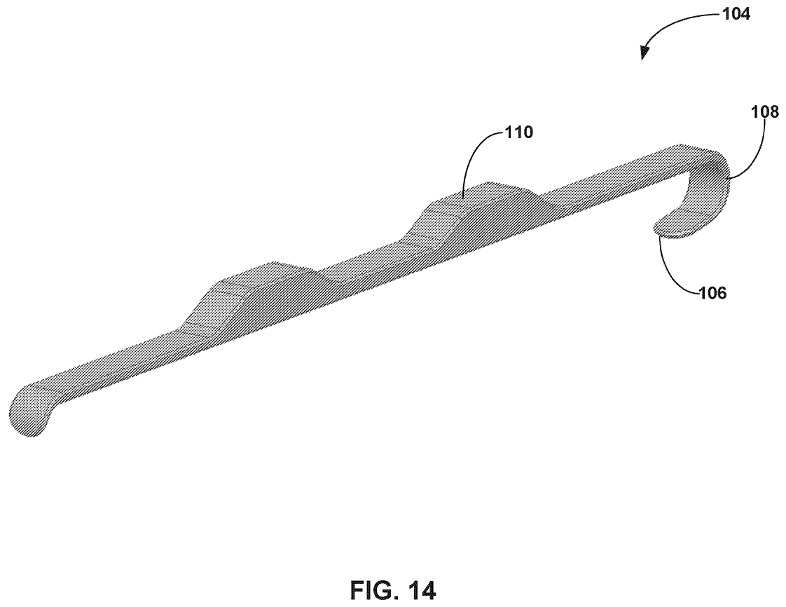

In practice, the clinician may utilize one or more retractors to pull cut skin 102 away from the incision line to help in large the surgical access area in addition to or in lieu of utilizing retraction guide 80. FIG. 14 illustrates one example configuration of a retractor 104 that may be utilized during a surgical procedure. Retractor 104 has a body defining the length terminating in a distal end 106. The distal end 106 of the retractor body can be bent back along the length of the body to define a curved (e.g., U-shaped) recess 108. In use, the distal end 106 of the retractor body can be inserted under the skin of the patient at the incision line, with the skin captured in recess 108, and the retractor pulled away from the incision line. To help the clinician grasp retractor 104 during use, the retractor may include one or more grips 110, such as regions of enlarged cross-sectional thickness.

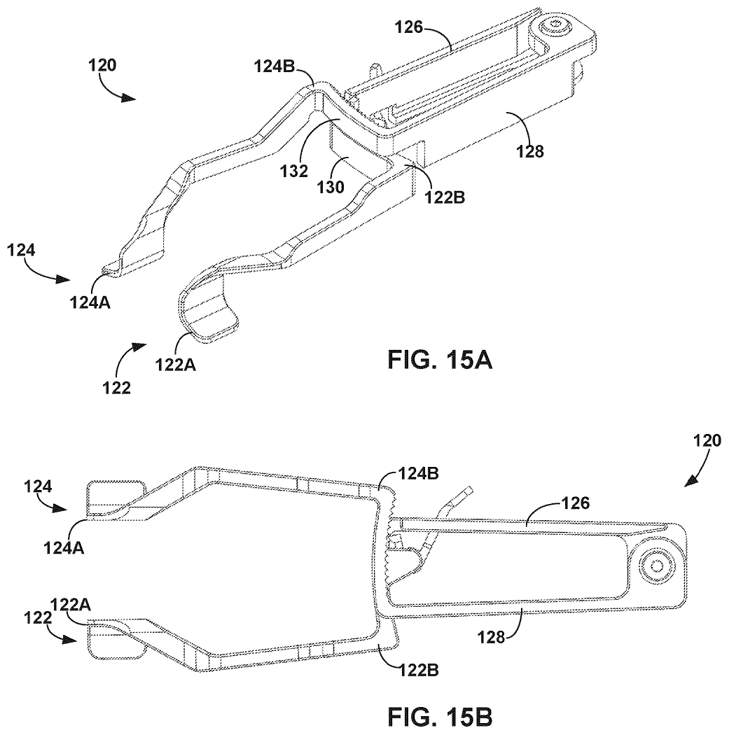

FIGS. 15A and 15B (collectively referred to as “FIG. 15”) are perspective and top view illustrations, respectively, of an exemplary retractor 120 that can be used during a surgical procedure according to aspects of the disclosure. Retractor 120 is illustrated as a locking retractor that includes a first arm 122 and a second arm 124 that are movable away from each other. First arm 122 is operatively connected to a first handle 126. Second arm 124 is operatively connected to a second handle 128. In particular, in the illustrated arrangement, first arm 122 is illustrated as extending from a first end 122A to a second end 122B, while second arm 124 is illustrated as extending from a first end 124A to a second end 124B. The second end 122B of first arm 122 is an on an opposite widthwise-side of retractor 120 than first handle 126, while the second end 124B of second arm 124 is on an opposite widthwise-side of the retractor than second handle 128.

To connect the first and second arms 122, 124 to corresponding first and second handles 126, 128, retractor 120 is illustrated as having first and second crossing arms 130, 132. First crossing arm 130 extends from the second end 122B of first arm 122 to first handle 126. Second crossing arm 132 extends from the second and 124B of second arm 124 to second handle 128. First and second cross arms 130, 132 extend across the width of retractor 120. Accordingly, when the first and second arms 122, 124 of the retractor 120 are not displaced relative to each other (e.g., the inner surface of the arms are adjacent to and/or in contact with each other), the first and second handles 126, 128 are positioned fully displaced away from each other. The clinician can grasp first and second handles 126, 128 and press the handles together, causing first and second arms 122, 124 to move away from each other. This can facilitate efficient, one-handed operation of retractor 120.

As briefly noted, a clinician may cut soft tissue and/or excise bone in a variety of different locations relative to the metatarsal being realigned to help mobilize the metatarsal for subsequent movement. In some examples, the clinician cuts soft tissue and/or excises bone in the TMT joint between the metatarsal being realigned and opposed cuneiform, such as TMT joint 230. Additionally or alternatively, the clinician may cut soft tissue in the joint space between the metatarsal being realigned and an adjacent metatarsal, such as in the intermetatarsal space between first metatarsal 210 and second metatarsal 212. The clinician can use any desired cutting instrument (e.g., saw, osteotome, reamer) with or without the aid of a resection guide to release soft tissue within the intermetatarsal space.

In some implementations, the clinician may utilize a biplanar tissue release instrument that is simultaneously positionable in the TMT joint space and the adjacent intermetatarsal space. For example, the clinician may utilize a cutting instrument that defines an angle or corner between two adjacent and connected cutting bodies. The clinician can insert the corner of the cutting instrument at a corner of the metatarsal, such as a proximal-lateral corner of the metatarsal. The clinician can position one cutting body of the biplanar tissue release instrument at least partially within the TMT joint space and a second cutting body of the biplanar tissue release instrument at least partially within the adjacent intermetatarsal space. The clinician can then advance the cutting instrument, e.g., by advancing the cutting instrument from a dorsal to a plantar direction, to cut soft tissue and/or otherwise release the corner of the metatarsal for realignment. Using a biplanar tissue release instrument can provide efficient and effective tissue release for the clinician, including when working through a comparatively small incision where it may be more difficult to visualize and access the two different adjacent joint spaces.

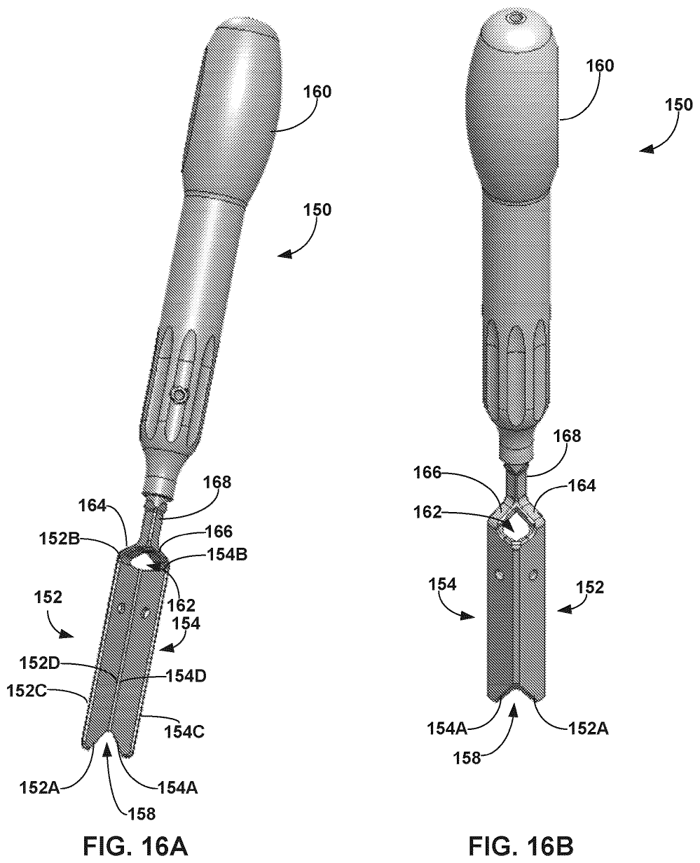

FIGS. 16A and 16B (collectively referred to as “FIG. 16”) are front and back perspective views, respectively, of an example biplanar tissue release instrument 150 that can be used during a surgical procedure, such as a minimally invasive surgical procedure as described herein. Instrument 150 is illustrated as having a first cutting body 152 and a second cutting body 154. First cutting body 152 is joined to a second cutting body 154 to define an angle 156 between the two cutting bodies. In the illustrated example of FIG. 16, angle 156 between first and second cutting bodies 152, 154 is shown as being approximately 90°, although different angles may be used in different configurations. In some examples, angle 156 may range from 45° to 135°, such as from 75° to 105°.

First cutting body 152 can have a length extending from a distal end 152A to a proximal end 152B. Similarly, second cutting body 154 can have a length extending from a distal end 154A to a proximal end 154B. First cutting body 152 can be connected to second cutting body 154 along an entirety of the length of the two cutting bodies or along only a portion of the lengths of the two cutting bodies.

In use, the distal ends 152A and 154A of the first and second cutting bodies 152, 154 can be advanced into adjacent joint spaces with a proximal portion of the cutting bodies trailing the leading distal ends advanced into the joint spaces. The distal end of biplanar tissue release instrument 150 (defined by the combination of the distal ends 152A and 154A of the first and second cutting bodies 152, 154) can have a variety of different profiles. In some examples, the distal end of instrument 150 is planar such than distal end 152A is in a same plane as distal end 154A across an entire width of the instrument. In other examples, the distal end of instrument 150 is nonplanar, e.g., that the distal ends 152A and 154A angle inwardly or outwardly relative to each other to define a groove/recess or projection. In the illustrated example of FIG. 16, instrument 150 is illustrated as defining a groove 158 between an inwardly angled distal end 152A of first cutting body 152 and an inwardly angled distal end 154A of second cutting body 154.

For example, first cutting body 152 can define a width extending from an outer edge 152C to an intersection location on an inner side 152D where the first cutting body intersects second cutting body 154. Second cutting body 154 can also define a width extending from an outer edge 154C to an intersection location on an inner side 154D where the second cutting body intersects first cutting body 152. The distal end 152A of first cutting body 152 can be angled inwardly along the width of the first cutting body, e.g., such that the length of the first cutting body is greater at the outer edge 152C of the cutting body than on the inner side 152D of the cutting body. The distal end 154A of first cutting body 154 can also be angled inwardly along the width of the second cutting body, e.g., such that the length of the second cutting body is greater at the outer edge 154C of the cutting body than on the inner side 154D of the cutting body.

By angling the distal ends 152A, 154A of the first and second cutting bodies 152, 154 inwardly, a groove 158 can be defined at the distal end of instrument 150. Groove 158 can be a space devoid of the material defining the cutting instrument extending at least partially along the length of the cutting instrument from the distal end of the instrument toward the proximal end of the instrument. Groove 158 may define a proximal-most apex where the distal ends 152A, 154A of the first and second cutting bodies 152, 154 converge at an intersection location between the two cutting bodies. Groove 158 may increase in size from the proximal-most apex of the groove to the distal-most end of biplanar tissue release instrument 150. In various implementations, groove 158 may generally define a V-shape as illustrated, a U-shape, a C-shape, or other suitable shape. By configuring biplanar tissue release instrument 150 with groove 158, the clinician may more precisely target soft tissue to be cut. The clinician can advance instrument 150 into the adjacent joint spaces and capture soft tissue targeted to be cut within groove 158 (e.g., using the downwardly extending apexes of the angled distal ends 152A, 152B of the first and second cutting bodies to funnel and/or otherwise direct targeted soft tissue into the groove).

Biplanar tissue release instrument 150 can be configured with one or more sharpen surfaces. A surface of the instrument can be sharpened by tapering the surface across the thickness of material defining the surface, e.g., through cutting, grinding, and/or other sharpening procedure, to form a surface that is sufficiently sharp to cut soft tissue during use. Biplanar tissue release instrument 150 can also be configured with one or more one surfaces, which can be surfaces that are not sharpen and are not designed or intended to cut soft tissue during use of the instrument.

In some implantations, the outer edge 152C of first cutting body 152 and the outer edge 154C of second cutting body 154 are each blunt. One or both of the distal end 152A of first cutting body 152 and the distal end 154A of second cutting body 154 may be sharpened along at least a portion of their length from a respective outer edge to an inner side where the two cutting bodies are joined together. This configuration may allow the clinician to insert instrument 150 into adjacent joint spaces, capturing soft tissue in groove 158 and cutting that tissue captured in the groove without cutting tissue contacted by the outer edge 152C of first cutting body 152 and the outer edge 154C of second cutting body 154.

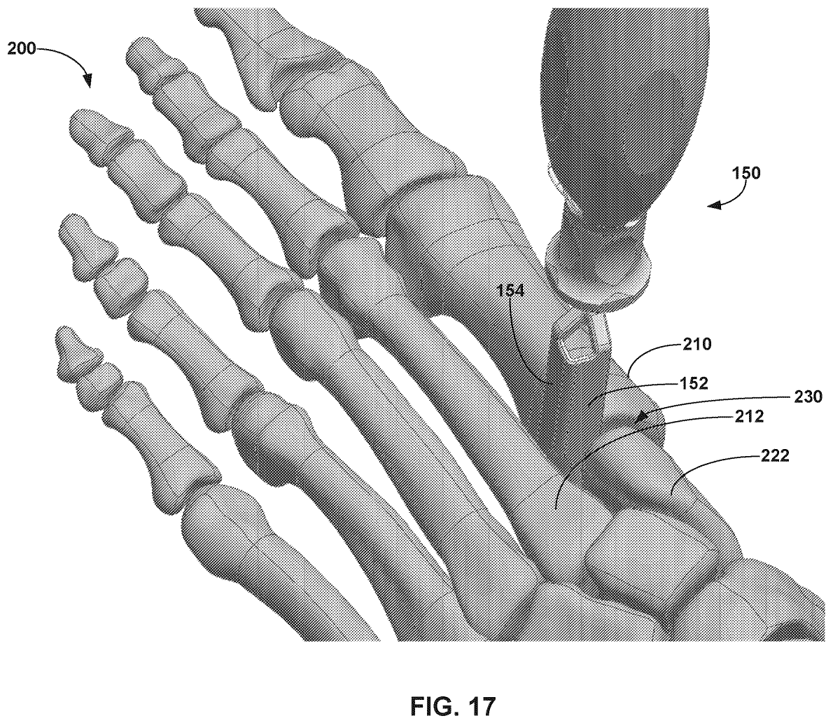

Biplanar tissue release instrument 150 can be configured (e.g., sized and shaped) to be positioned in adjacent joint spaces. For example, during use, the clinician can position first cutting body 152 in TMT joint 230 and position second cutting body 154 and the joint space between first metatarsal 210 and second metatarsal 212. FIG. 17 is a perspective illustration of foot 200 showing an example configuration of biplanar tissue release instrument 150 inserted at a proximal-lateral corner of first metatarsal 210, e.g., with the proximal-lateral corner of the metatarsal in the angular space defined between the intersection of first cutting body 152 and second cutting body 154. Instrument 150 can be inserted through a comparatively small incision in the skin of the patient, as discussed above, to help mobile first metatarsal 210 for subsequent repositioning. The clinician can advance biplanar tissue release instrument 150 through dorsal incision in a plantar direction. As the clinician advances the instrument simultaneously into the two adjacent joint spaces, the clinician can capture target soft tissue in the groove 158 between first cutting body 152 and second cutting body 154. One or more sharpen surfaces defining groove 158 can cut the soft tissue directed into the groove by the clinician while the outer edges of the cutting bodies are inserted into the adjacent joint spaces without significantly cutting soft tissue along the outer edges.

While the specific dimensions of biplanar tissue release instrument 150 can vary, in some implementations, first cutting body 152 and second cutting body 154 each have a length ranging from 20 mm to 60 mm, such as from 25 mm to 55 mm, from 30 mm to 50 mm, from 35 mm to 45 mm, or approximately 40 mm (e.g., plus or minus 10%). In addition, first cutting body 152 and second cutting body 154 may have a width ranging from 4 mm to 10 mm, such as from 5 mm to 9 mm, from 6 mm to 8 mm, or approximately 7 mm (e.g., plus or minus 10%). In some examples, one or both of first cutting body 152 and second cutting body 154 may include a depth indicator that indicates how deep the distal end of the cutting body has been inserted into a space. For example, as illustrated, first cutting body 152 and second cutting body 154 each include an aperture extending through the planar faces of the cutting bodies. The aperture may be at a defined location along the length of the cutting body corresponding to a particular distance between the distal tip of the cutting body and the depth indicator (e.g., at 20 mm, 25 mm, 30 mm, 35 mm, 40 mm). In some examples, the depth indicator includes a projection (e.g., depth step) configured to contact a dorsal surface of bone and/or skin to limit how deep the cutting body can be inserted into the joint space. For example, when the depth indicator is an aperture, a wire can be inserted through the aperture to provide a physical stop when the cutting body has been advanced to a location where the wire inserted through the aperture contacts skin and/or bone.

First cutting body 152 and second cutting body 154 can be integrally and permanently joined together to form a monolithic pair of cutting bodies. For example, first cutting body 152 and second cutting body 154 may be formed as a unitary structure (e.g., through casting or milling). In other examples, first cutting body 152 and second cutting body 154 are formed as separate structures that are then permanently joined together (e.g., through welding) or joined together but separable from each other (e.g., through mechanical locking and/or other temporary fixation structures).

With further reference to FIG. 16, biplanar cutting instrument 150 is illustrated as including a handle 160. In the illustrated configuration, a clinician can manually grasp handle 160 to control instrument 150 and direct the instrument into the target adjacent joint spaces. In some examples, handle 160 is operatively connected to first cutting body 152 and second cutting body 154 without being configured to be removed during use. In other examples, handle 160 is operatively connected to first cutting body 152 and second cutting body 154 and can be removed by the clinician during the surgical procedure. For example, the clinician may insert biplanar cutting instrument 150 into adjacent joint spaces and remove handle 160 from the cutting bodies, leaving the cutting bodies in the joint spaces. This may provide better visualization of the cutting bodies in the joint spaces for the clinician, e.g., when viewing directly with the unaided eye by the clinician and/or when viewing under fluoroscopic imaging. Biplanar tissue release instrument 150 may additionally or alternatively be connectable to a handle of a powered drive unit, such as a reciprocating hand unit controllable by the clinician. For example, biplanar tissue release instrument 150 may include a stem having an AO connection that can be engaged with and electrically or pneumatically powered drive unit.

In the illustrated configuration of FIG. 16, biplanar tissue release instrument 150 defines an opening 162 between first cutting body 152 and second cutting body 154 at the proximal ends of the cutting bodies. Opening 162 can be located at the proximal end of the intersection between first cutting body 152 and second cutting body 154. When so configured, biplanar tissue release instrument 150 may be devoid of a planar surface intersecting the axis defined the longitudinally extending extent of the intersection between the first and second cutting bodies 152, 154. Configuring biplanar tissue release instrument 150 with a proximal opening aligned with the intersection between first cutting body 152 and second cutting body 154 may be useful for a variety of reasons. Opening 162 can help prevent the accumulation of bodily matter in a corner pocket of the instrument that may otherwise occur, helping with cleaning and sterilization of instrument 150 between uses. Additionally or alternatively, opening 162 may provide the clinician with a visualization opening to see the positioning of the instrument during insertion and/or after insertion. For example, the clinician may view the positioning of the instrument with the unaided eye and/or under fluoroscopy (optionally with handle 160 removed during viewing) to confirm that the instrument is appropriately inserted at the desired location (e.g., proximal-lateral corner of the metatarsal) and to all repositioning if not inserted at the desired location.

In the illustrated example, instrument 150 is implemented with a first arm 164 extending from first cutting body 152 to handle 160 and a second arm 166 extending from a second cutting body 154 to the handle. First arm 164 can extend from the proximal end 152B of first cutting body 152 to a stem 168 to which handle 160 is attached. Second arm 166 can extend from the proximal end 154B of second arm 154 to stem 168 to which handle 160 is attached. Opening 162 can be defined as a void space between first arm 164 and second arm 166. In some examples, opening 162 defines an area ranging from 20 mm² to 250 mm², such as from 50 mm² to 150 mm².

Independent of whether the clinician performs any steps to mobilize the metatarsal for repositioning or the specific types of instrument(s) the clinician uses to help mobilize the bone, the clinician can prepare the opposed ends of the bones (e.g., first metatarsal 210 and medial cuneiform 222) for fusion. In general, the clinician can prepare the end of each bone forming a TMT joint so as to promote fusion of the bone ends across the TMT joint following realignment. Bone preparation may involve using a tissue removing instrument to apply a force to the end face of the bone so as to create a bleeding bone face to promote subsequent fusion. Example bone preparation instruments that can be used (which may also be referred to as tissue removing instruments) include, but are not limited to, a saw, a rotary bur, a rongeur, a reamer, an osteotome, a curette, and the like. The bone preparation instrument can be applied to the end face of the bone being prepared to remove cartilage and/or bone. For example, the bone preparation instrument may be applied to the end face to remove cartilage (e.g., all cartilage) down to subchondral bone. Additionally or alternatively, the bone preparation instrument may be applied to cut, fenestrate, morselize, and/or otherwise reshape the end face of the bone and/or form a bleeding bone face to promote fusion. In instances where a cutting operation is performed to remove an end portion of a bone, the cutting may be performed freehand or with the aid of a cutting guide having a guide surface positionable over the portion of bone to be cut. When using a bone preparation guide, a cutting instrument can be inserted against the guide surface (e.g., between a slot define between two guide surfaces) to guide the cutting instrument for bone removal.

In some examples, the clinician cuts at least one bone defining the TMT joint 230 (e.g., one or both of first metatarsal 210 and medial cuneiform 222). The clinician may cut both bones defining the TMT joint or may cut only one bone defining the joint and perform a different preparation technique on the other bone. The clinician may realign the metatarsal in one or more planes before preparing both end faces, after preparing the end face of one bone but not the other bone, and/or after preparing the end faces of both bones. Accordingly, unless otherwise specified, the order of bone preparation and/or movement is not limited.

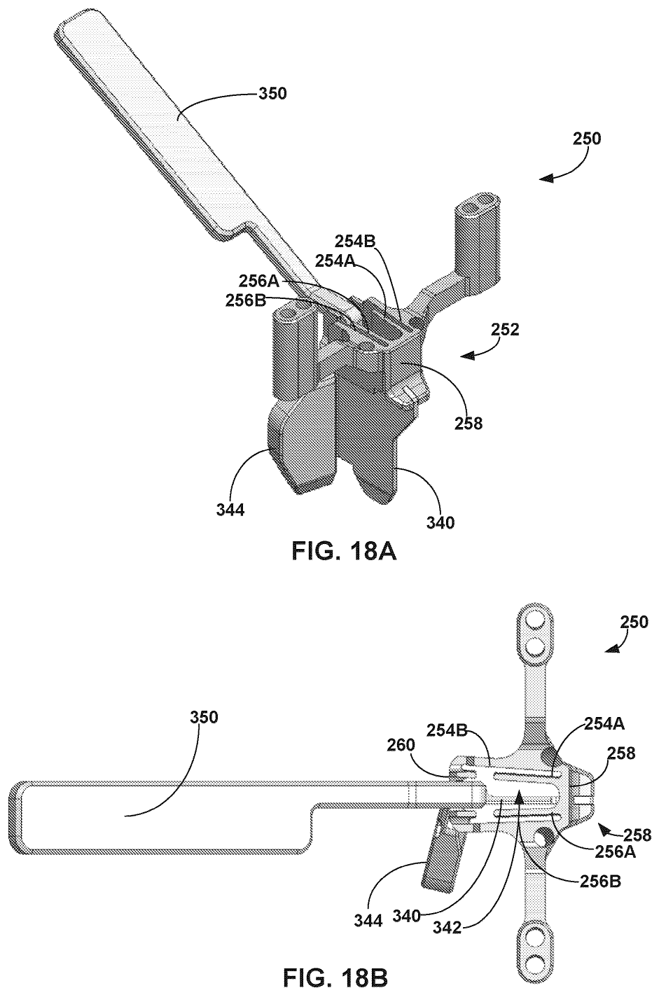

With reference to FIG. 4, the example technique involves positioning at least one guide surface of a bone preparation guide over a bone to be cut (step 16 on FIG. 4) and using the bone preparation guide to guide a bone preparation instrument to prepare the end of the bone (step 18 on FIG. 4). A variety of different bone preparation guides can be used to guide a bone preparation instrument. FIGS. 18A and 18B (collectively referred to as “FIG. 18”) illustrate one example bone preparation guide 250 that may be used as part of a minimally invasive procedure. FIG. 18A is a perspective view of bone preparation guide 250. FIG. 18B is a top view of bone preparation guide 250. In some configurations, as will be described in greater detail below, bone preparation guide 250 may include one or more guide surfaces sized smaller than a bone to be prepared using the guide surface. The comparatively small guide surface may facilitate insertion of bone preparation guide 250 into a comparatively small incision 100 without necessitating a larger incision to accommodate a full size bone preparation guide that includes a guide surface covering an entirety of the bone to be prepared.

In the illustrated example of FIG. 18, bone preparation guide 250 includes a body 252 that defines at least one guide surface 254 positionable over a side of the bone to be prepared, such as a dorsal side or a medial side of the bone to be prepared. The clinician can place a bone preparation instrument adjacent to, and optionally in contact with, the guide surface and translate the bone preparation instrument relative to the guide surface to perform a cut in a plane parallel to the guide surface. For example, the clinician may place the bone preparation instrument in contact with the guide surface and then translate the cutting instrument relative to the guide surface, e.g., plantarly into a bone and/or in a medial or lateral direction. The guide surface may bound movement of the bone preparation instrument to a desired preparation plane.

In some configurations, bone preparation guide 250 defines a single guide surface. Single guide surface can be positioned over a bone to be prepared, such as metatarsal 210 and/or cuneiform 222. For example, the single guide surface can be positioned over an end of a first bone to be prepared and, after preparing the end of the first bone using the guide surface, repositioned over an end of a second bone to be prepared. The single guide surface can then be used again to guide a bone preparation instrument to prepare the end of the second bone.

In other examples, bone preparation guide 250 may include multiple guide surfaces spaced apart from each other. For example, bone preparation guide 250 may include at least one guide surface positionable over a first bone to be prepared and at least one other guide surface positionable over a second bone to be prepared, where the two bones are spaced apart from each other (e.g., by a joint separating the two bones). In the illustrated example of FIG. 18, for instance, bone preparation guide 250 includes a first guide surface 254A positionable over an end of the first bone to be prepared (e.g., metatarsal 210) and a second guide surface 256A positionable over an end of a second bone to be prepared (e.g., cuneiform 222). Bone preparation guide 250 may include multiple guide surfaces (e.g., multiple slots) positionable the first bone and/or second bone, with different guide surfaces being spaced from each other along the length of the bone. This can provide the clinician with options to select how far along the length of the bone the clinician wishes to cut or otherwise prepare the bone end.

In some configurations, one or more of the guide surfaces defined by the body 252 of bone preparation guide 250 are open on an opposed side (e.g., such that the bone preparation instrument is not constrained between two opposed surfaces. In other configurations, such as that illustrated in FIG. 18, bone preparation guide 250 includes a facing guide surface spaced apart from a corresponding guide surface to define a slot between the two guide surfaces. In particular, in the illustrated example, bone preparation guide 250 includes a first facing guide surface 254B in a second facing guide surface 256B. First facing guide surface 254B is positioned adjacent to first guide surface 254A to define a slot between the two guide surfaces. Similarly, second facing guide surface 256B is positioned adjacent to second guide surface 256A to define a slot between the two guide surfaces. Each respective slot can be sized to allow a bone preparation instrument to be inserted through the slot to guide the bone preparation instrument for preparing an underlying bone.

When bone preparation guide 250 includes at least two guide surfaces (e.g., slots) positionable over different bones separated from each other (e.g., metatarsal 210 and cuneiform 222) the guide surfaces can be parallel to each other or can be angled relative to each other in the transverse plane and/or to the end face of the corresponding bone to be prepared. Further, each guide surface can extend straight (e.g., parallel) or an angle in a dorsal to plantar direction (in other words, in the sagittal plane) and can guide the bone preparation instrument in a direction defined by the guide surface.

In some configurations of bone preparation guide 250, the one or more guide surfaces 254, 256 of the bone preparation guide are sized relative to the expected size of the one or more bones to be prepared using the guide surface. For example, bone preparation guide 250 may include one or more guide surfaces sized smaller than the size of the bone to be prepared using the guide surface. The one or more guide surfaces can have a length that does not span the entire width of the bone to be prepared using the guide surface but, instead, only covers a portion of the width of the bone to be prepared using the guide surface.

For example, the body 252 of bone preparation guide 250 can have a length extending from a first end 258 to a second end 260. The length of the body 252 of bone preparation guide 250 can be aligned (e.g., generally parallel) to the lengthwise direction of the one or more guide surfaces defined by the bone preparation guide. In use, the lengthwise extent of bone preparation guide 250 can be placed over the widthwise extent (e.g., diameter) of one or more bones to be prepared.

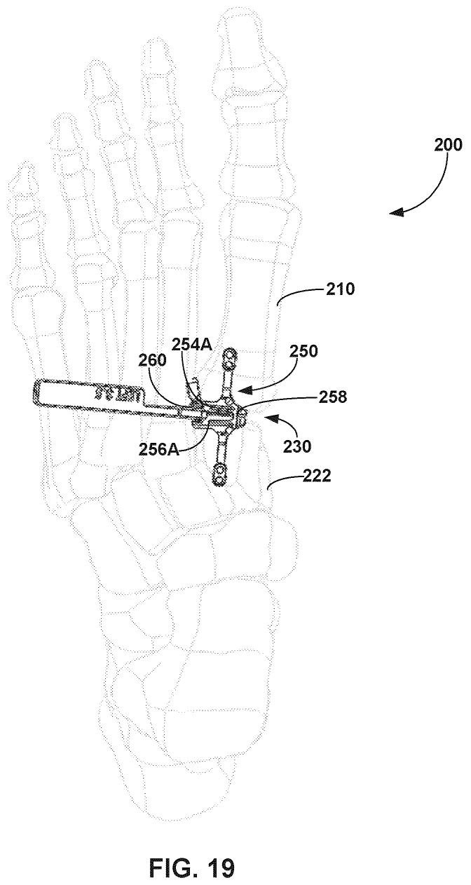

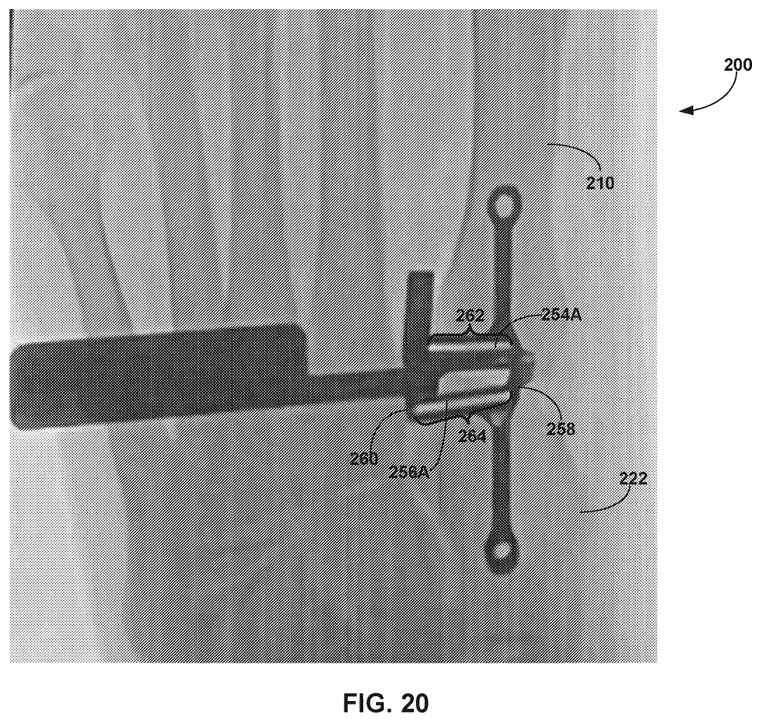

FIG. 19 is a dorsal view of foot 200 illustrating an example configuration and positioning of bone preparation guide 250 relative to one or more bones to be prepared using the guide. FIG. 20 is a dorsal view radiographic image of an example foot 200 showing an example configuration and positioning of bone preparation guide 250 relative to one or more bones to be prepared using the guide. As illustrated in the examples of FIGS. 19 and 20, each of the guide surfaces 254A, 256A defined by the bone preparation guide are sized smaller than the width (e.g., diameter) of the bones to be prepared using the guide surfaces. In particular, first guide surface 254A is illustrated as having a length between the first and second ends 258, 260 of the bone preparation guide less than the diameter of first metatarsal 210. Second guide surface 256A is illustrated as having a length between the first and second ends 258, 260 of the bone preparation guide less than the diameter of medial cuneiform 222. When configured with corresponding facing guide surfaces, each facing guide surface may have a length substantially the same as that guide surface that the facing guide surface faces.

Configuring bone preparation guide 250 with one or more guide surfaces sized shorter than the extent of the one or more corresponding bones to be prepared using the guide surface can help minimize the size of the incision 100 made through the skin 102 of the patient to surgically access the bones. In some examples, incision 100 is sufficiently small that the skin 102 of the patient cannot be retracted to expose the full widthwise extent (e.g., diameter) of the one or more bones to be prepared through the incision and/or the skin cannot be sufficiently retracted to insert a bone preparation guide with one or more guide surfaces spanning the full widthwise extent of the one or more bones to be prepared. In these and other examples, utilizing bone preparation guide 250 with one or more guide surfaces shorter than the extent of the one or more corresponding bones to be prepared using the guide surface can help implement a surgical procedure with reduced incision length.

First guide surface 254A (and/or slot defined by the guide surface) can define a first length 262 (FIG. 20) from the first end 258 to the second end 260. Second guide surface 256A (and/or slot defined by the guide surface) can define a second length 264 (FIG. 20) from the first end 258 to the second end 260. The first length 262 of first guide surface 254A (and/or slot defined by the guide surface) can be sized less than the diameter of a first bone to be prepared using the guide surface (e.g., first metatarsal 210 and/or media cuneiform 222). The second length 264 of the second guide surface 256A (and/or slot defined by the guide surface) can be sized less than the diameter of a second bone to be prepared using the guide surface (e.g., medial cuneiform 222 and/or first metatarsal 210). In some configurations, each of the one or more guide surfaces defined by bone preparation guide 250 have a length less than the diameter of each of the bones to be prepared using the bone preparation guide.

In practice, the diameter of the bones of the patient undergoing the procedure using bone preparation guide 250 may vary patient-to-patient. Accordingly, when sizing the one or more guide surfaces relative to the diameter of one or more bones of a patient, an average bone size determined from a representative population set can be used to establish average sizing of the one or more bones. The one or more guide surfaces can then be sized relative to this average bone sizing determined from the representative data set.

In other examples, bone preparation guide 250 (as well as any other instruments and/or implants used during the procedure) can be designed and constructed with patient-specific sizing and/or characteristics (e.g., one or more characteristics configured to interface with patient-specific anatomical attributes). In these examples, the anatomical characteristics (e.g., size and/or shape) of at least a portion of the patient's foot undergoing the procedure can be determined prior to performing the surgical procedure. The patient's foot may be imaged to provide data indicative of the size and structure of the patient's foot. A computational model representative of the patient's foot may then be generated and bone preparation guide 250 and/or other instruments and/or implants to be used during a surgical procedure sized and/or otherwise configured to the specific anatomical characteristics of the foot of the patient undergoing the procedure. The instruments and/or implants can then be manufactured to provide one or more patient-specific components that are then used during the subsequent surgical procedure.

In either case, in some implementations, bone preparation guide 250 has at least one guide surface sized relative to the diameter of bone to be prepared using the guide surface. For example, bone preparation guide 250 may include first guide surface 254A (and the corresponding slot defined thereby) having length 262 less than the diameter of first metatarsal 210 (where the diameter is measured at the location over which the guide surface and/or slot is positioned). Bone preparation guide 250 may additionally or alternatively include second guide surface 256A (and the corresponding slot defined thereby) having length 264 less than the diameter of medial cuneiform 222 (where the diameter is measured at the location over which the guide surface and/or slot is positioned). In various examples, the length 262, 264 of the guide surfaces (and the corresponding slots defined thereby) can be less than 80% of the diameter of the underlying bone(s) to be prepared using the guide surfaces, such as less than 70% of the diameter, less than 60% of the diameter, or less than 50% of the diameter. For example, the length 262, 264 of the guide surfaces may be within a range from 25% to 75% of the diameter of the underlying bone(s) to be prepared using the guide surfaces, such as within a range from 40% to 60% of the diameter.

The lengths 262, 264 of one or more (e.g., each) of the guide surfaces defined by bone preparation guide 250 (and the corresponding slots defined thereby) may be less than 30 cm, such as less than 25 cm, or less than 20 cm. In some examples, the lengths 262, 264 of one or more (e.g., each) of the guide surfaces defined by bone preparation guide 250 (and the corresponding slots defined thereby) may be within a range from 8 cm to 25 cm, such as from 10 cm to 20 cm, from 12 cm to 18 cm, or from 13 cm to 16 cm. For example, the lengths 262, 264 of one or more (e.g., each) of the guide surfaces defined by bone preparation guide 250 (and the corresponding slots defined thereby) may be approximately 15 cm (±10%). When configured with multiple guide surfaces such as in the illustrated example, each guide surface (and/or the corresponding slot defined thereby) may have the same length, or one guide surface (and the corresponding slot defined thereby) may have a length greater than another guide surface (and the corresponding slot defined thereby).

As initially introduced in connection with FIG. 4, the example surgical technique can involve using bone preparation guide 250 to guide a bone preparation instrument to prepare the end of the bone (step 18 on FIG. 4). When bone preparation guide 250 is configured with one or more guide surfaces sized smaller than the size of the bone to be prepared (e.g., smaller than the widthwise extent of the bone in the transverse plane), the clinician may manipulate the bone preparation instrument to prepare the bone beyond one or more boundaries defined by the bone preparation guide. For example, the clinician may manipulate the bone preparation instrument to prepare the end of the bone directly underlying the guide surface and/or slot defined thereby. The clinician may also manipulate the bone preparation instrument to prepare the end of the bone outside (e.g., medial of and/or lateral of) the guide surface and/or slot defined thereby.

In some examples, the clinician may translate the bone preparation instrument along the length of the guide surface and/or slot defined thereby to prepare that portion of the bone directly underlying the guide surface and/or slot defined thereby. The clinician may additionally angle the bone preparation instrument to extend the instrument beyond the first end 258 and/or the second end 260 of bone preparation guide 250. The clinician can angle the bone preparation instrument and guide the instrument to extend beyond one or more side boundaries of bone preparation guide 250 before, during, and/or after advancing the bone preparation instrument along the path (e.g., linear path) defined by the length of the guide surface and/or slot defined thereby. For example, with the bone preparation instrument aligned with a guide surface of bone preparation guide 250 (e.g., in contact with the guide surface, in a slot bounded by the guide surface), the clinician can change the bone preparation instrument from being generally perpendicular with respect to the length of the guide surface to being at a non-perpendicular angle with respect to the length of the guide surface. This may cause the proximal end of the bone preparation instrument to shift laterally in the distal end of the bone preparation instrument to shift medially relative to a perpendicular position, or vice versa.

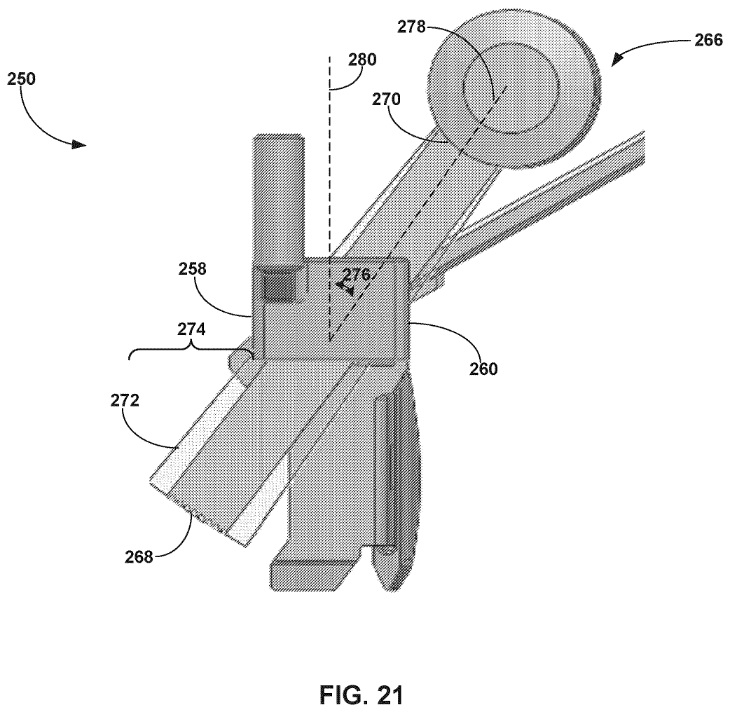

FIG. 21 is a side view of an example configuration of bone preparation guide 250 illustrating an example bone preparation instrument 266 guided at an angle relative to a guide surface and extending beyond an end of the bone preparation guide. In particular, FIG. 21 illustrates an example bone preparation instrument 266 in the form of a saw blade. The saw blade 266 has a length extending from a distal end 268 to a proximal end 270. The proximal end 270 can be attached to a handle graspable by the clinician or a powered hand unit manipulable by the clinician (e.g., an oscillating or reciprocating hand unit operating under pneumatic or electrical power. FIG. 21 illustrates an example oscillating motion pattern 272 the saw blade 266 can travel during operation of the bone preparation instrument.

As shown in the example of FIG. 21, bone preparation instrument 266 can extend beyond one or both ends 258, 260 of the body defining bone preparation guide 250. For example, bone preparation instrument 266 can include a distal portion 274 that can be angled to extend beyond one or both ends 258, 260 of bone preparation guide 250 which, in the illustrated arrangement, is shown as extending beyond first end 258 of the bone preparation guide. Bone preparation instrument 266 can be guided at an angle relative to a perpendicular axis extending along the height of the bone preparation instrument (e.g., in the sagittal plane). As a result, distal portion 274 of bone preparation instrument 266 can extend under an end of bone preparation guide 250 to prepare a portion of bone located outside of the bone preparation guide (e.g., located outside the region directly under a guide surface of the bone preparation guide).

The extent of bone located beyond one or both ends 258, 260 of bone preparation guide 250 that can be prepared by guiding bone preparation instrument at an angle can vary, e.g., based on the length of the bone preparation instrument, the depth to which the bone preparation instrument is inserted into the bone preparation guide (e.g., along a guide surface and/or through a slot), and/or the angle at which the bone preparation instrument is guided. Bone preparation instrument 266 can be angled at an angle 276 defined between an axis 278 bisecting the length of the bone preparation instrument and an axis 280 perpendicular to the length of the one or more guide surfaces and/or parallel to the height of the bone preparation instrument. In some examples, angle 276 may be within a range from 10° to 80°, such as from 15° to 75°, from 20° to 70°, or from 30° to 60°. Angle 276 may be measured in either the positive or negative direction relative to axis 280 depending on which way clinician is angling the bone preparation instrument.

In practice, the clinician may not advance bone preparation instrument 266 beyond an end of bone preparation guide 250 at a single angle but, instead, may sweep the bone preparation instrument back and forth over a range of angles which may include, or encompass, any of the foregoing mention angles. In different implementations, the clinician may advance a distal portion 274 of bone preparation instrument 266 beyond a single end of bone preparation guide 250 (e.g., a medial end such that the bone preparation instrument prepares a portion of bone located medially beyond the end of the bone preparation guide or a lateral end such that the bone preparation instrument prepares a portion of bone located laterally beyond the end of the bone preparation guide) or beyond both ends of the bone preparation guide.

During a surgical procedure, the clinician may translate bone preparation instrument 266 along the length of a guide surface defined by the bone preparation instrument to prepare a portion of the bone underlying the guide surface. For example, the clinician may cut a portion of the bone underlying the guide surface by guiding a cutting instrument along the length of the guide surface. The clinician may move bone preparation instrument 266 linearly along the length of the guide surface (e.g., slot defined by the guide surface), either in a single direction or a back-and-forth direction. When translating the bone preparation instrument 266 along the guide surface, the lengthwise axis 278 of the bone preparation instrument may be parallel to axis 280 or may be angled relative to the axis (e.g., at any of the foregoing described angles).

Before, during, and/or after translating the bone preparation instrument along the guide surface to prepare that portion of the bone directly under the guide surface (e.g., cut slot defined by the guide surface), the clinician can advance the distal portion 274 of the bone preparation instrument beyond one or both ends of bone preparation guide 250 to that portion of bone located beyond the one or both ends. Accordingly, the clinician may translate bone preparation instrument 266 and then change the angle of the bone preparation instrument to advance the instrument beyond one or both ends of bone preparation guide 250, or the clinician may translate the bone preparation instrument at an angle in advance the angled instrument beyond one or both ends of the bone preparation guide.

With further reference to FIGS. 18A and 18B, the body defining bone preparation guide 250 includes a first sidewall defining the first end 258 of the body and a second sidewall defining the second end 260 of the body. When bone preparation guide 250 is positioned through an incision on a dorsal side of one or more bones to be prepared using the guide, the first sidewall 258 defining the first end of the body can be positioned on a medial side and the second sidewall 260 defining the second end of the body can be positioned on a lateral side.

When advancing bone preparation guide 266 beyond the first and/or second end of the body of bone preparation guide 250, the distal portion of the bone preparation guide may project under the bottom of the first sidewall 258 and/or the bottom of the second sidewall 260. To increase the angle at which bone preparation instrument 266 can be angled relative to bone preparation guide 250 and/or the extent of bone that can be prepared beyond the bone preparation guide, one or both sidewalls 258, 260 may include one or more cut outs into which the bone preparation instrument can be angularly inserted. The one or more cut outs can be recesses, grooves, and/or other areas of in which the sidewall of the bone preparation guide is absent (e.g., and bounded by an adjacent region of sidewall). The bone preparation instrument can be advanced at least partially into the one or more cutouts, e.g., causing at least a portion of the bone preparation guide to extend within the region of the bone of the bone preparation guide otherwise delimited by the one or more sidewalls. Configuring bone preparation guide 250 with one or more wall cutouts can allow bone preparation instrument 266 to be guided at a greater angle 276 as compared to if the bone preparation guide were to contact a top and/or bottom edge of a wall surface in the absence of the one or more cutouts. As a result, the distal portion of bone preparation instrument 266 may be advanced farther medially and/or laterally relative to the medial and/or lateral sides of bone preparation guide 250, preparing a greater extent of bone located beyond the medial and/or lateral sides of the guide than if the cutouts were not present.

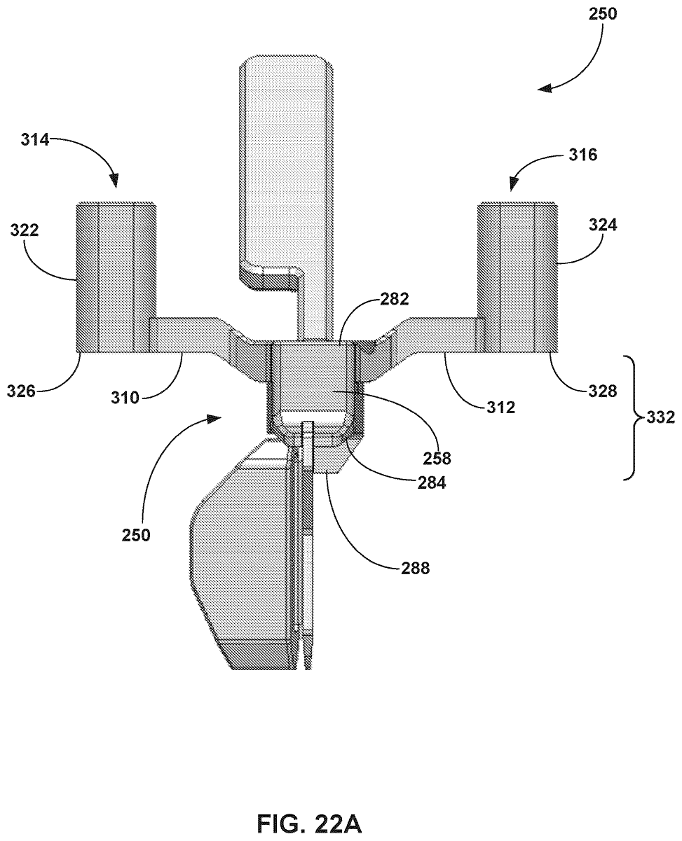

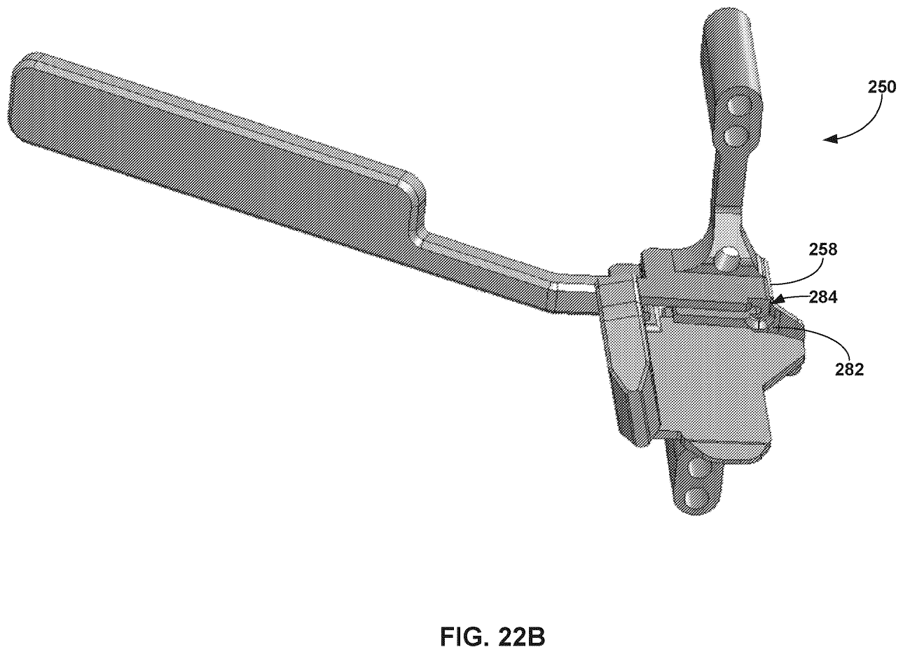

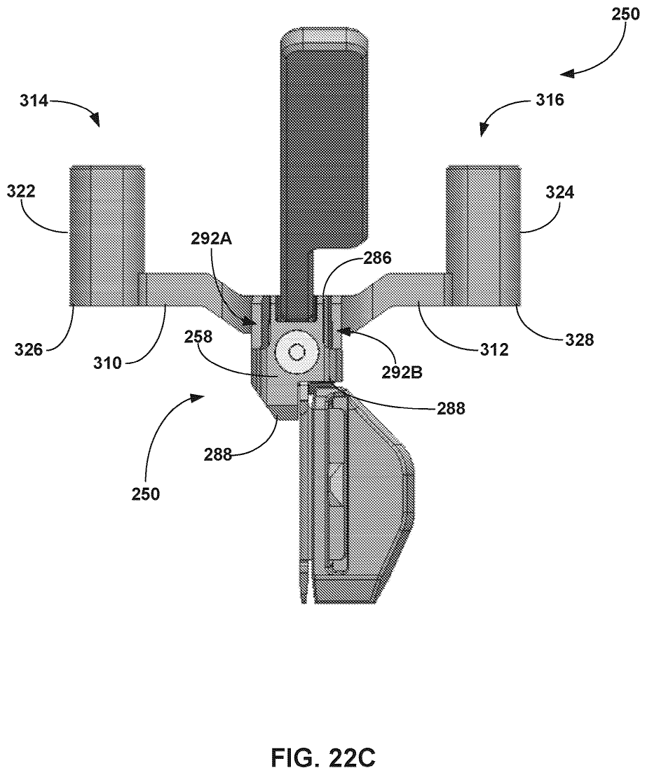

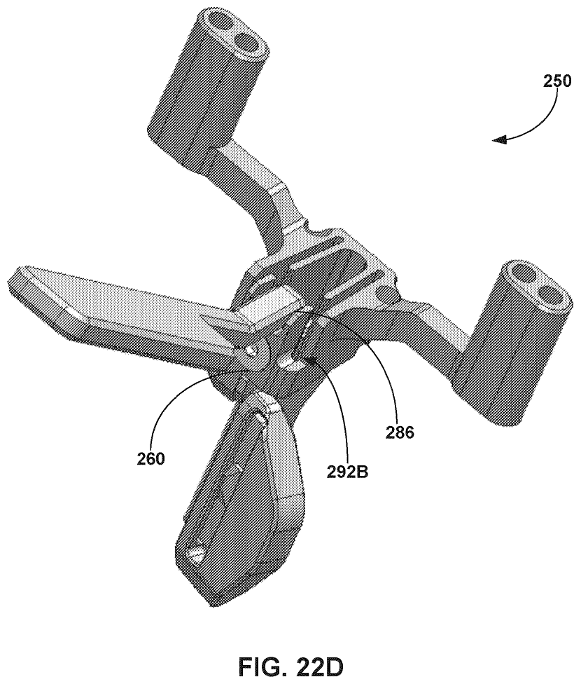

FIGS. 22A-22D (collectively referred to as “FIG. 22”) are different views of an example configuration of bone preparation guide 250 illustrating example sidewall cutouts that may be utilized. FIG. 22A is medial side view of bone preparation guide 250. FIG. 22B is a bottom perspective view of bone preparation guide 250. FIG. 22C is a lateral side view of bone preparation guide 250. FIG. 22D is a top-lateral perspective view of bone preparation guide 250.

In the example of FIG. 22, bone preparation guide 250 includes first sidewall 258 bounding one side of the one or more guide surfaces (e.g., one or more slots) and second sidewall 260 bounding an opposite side of the one or more guide surfaces (e.g., one or more slots). As illustrated in FIGS. 22A and 22B, first side wall 258 can extend from a top end 282 to a bottom end 284. As illustrated in FIGS. 22C and 22D, second sidewall 260 can extend from a top end 286 to a bottom end 288. First and/or second sidewalls 258, 260 can include one or more cut outs relative to the top and/or bottom ends of the sidewall.

For example, first side wall 258 may include a first sidewall cutout 290 extending from bottom end 284 toward the top end 282 of the sidewall. First sidewall cutout 290 can be aligned with a guide surface (e.g., slot) defined by bone preparation guide 250. When configured with multiple guide surfaces, bone preparation guide 250 may include multiple sidewall cutouts (e.g., each configured as described with respect to first sidewall cutout 290), with each cutout being aligned with a respective guide surface and/or slot.

Second sidewall 260 may additionally or alternatively include a second sidewall cutout 292, which is illustrated as a pair of second side wall cutouts 292A, 292B. Each second sidewall cutout 292 may extend from top end 286 toward the bottom end 288 of the sidewall. Second sidewall cutout 292 can be aligned with a guide surface (e.g., slot) defined by bone preparation guide 250. When configured with multiple guide surfaces, bone preparation guide 250 may include multiple sidewall cutouts (e.g., each configured as described with respect to second sidewall cutout 292), with each cutout being aligned with a respective guide surface and/or slot.

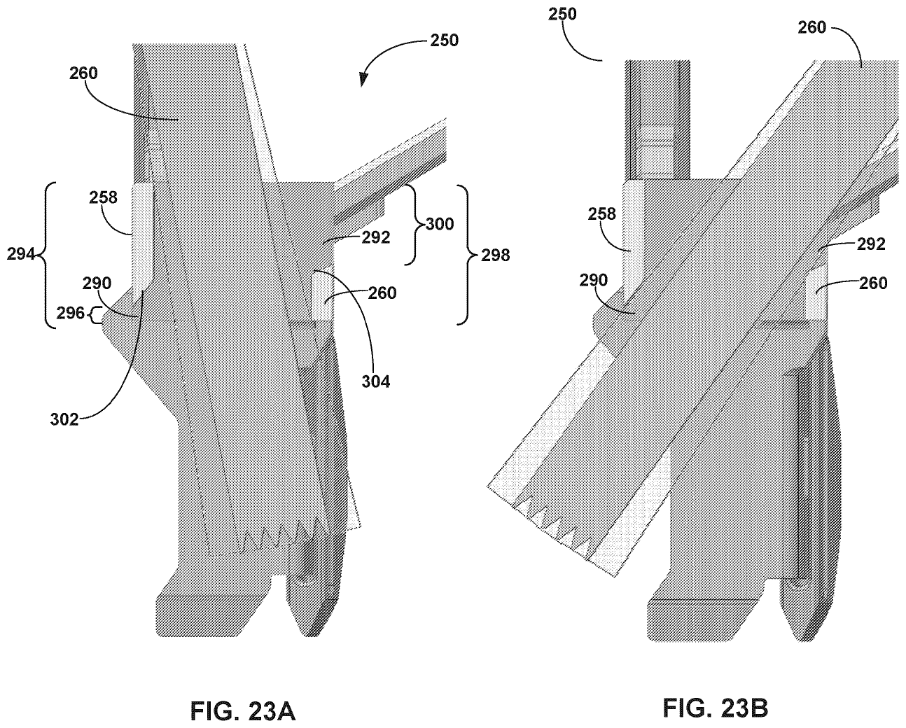

FIGS. 23A and 23B are sectional side views of bone preparation guide 250 illustrating example sidewall cutout configurations that can be used on the bone preparation guide. FIG. 23A illustrates bone preparation instrument 266 aligned with a guide surface of bone preparation guide 250 (e.g., inserted into a slot of the bone preparation guide) in a first angular orientation. FIG. 23B illustrates bone preparation instrument 266 aligned with the guide surface of bone preparation guide 250 in a second angular orientation, with a distal portion of the bone preparation instrument extending beyond the end (e.g., sidewall) of the bone preparation guide.

As illustrated, first sidewall 258 can define a height 294 measured from the top end to the bottom end of the sidewall. First sidewall cutout 290 can also define a height 296. In some implementations, the height 296 of first sidewall cutout 290 is less than 50% of the overall height 294 of first sidewall 258, such as less than 40%, less than 30%, less than 25%, or less than 20%. For example, the height 296 of first sidewall cutout 290 may be within a range from 10% to 50% of the overall height 294 of first sidewall 258, such as from 20% to 40%. The height 296 of first sidewall cutout 290 may be sufficiently large to allow bone preparation instrument 266 to advance to a desired location under the sidewall. However, the height 296 of first sidewall cutout 290 may be sufficiently small to restrict the angle at which the bone preparation instrument 266 can be advanced under the sidewall (e.g., to help prevent unintended cutting in a medial direction).

FIGS. 23A and 23B also illustrate that second sidewall 260 can define a height 298 measured from the top end to the bottom end of the sidewall. Second sidewall cutout 292 can also define a height 300. In some implementations, the height 300 of second sidewall cutout 292 is greater than 25% of the overall height 298 of second sidewall 260, such as greater than 30%, greater than 40%, greater than 50%, greater than 60%, or greater than 75%. For example, the height 300 of second sidewall cutout 292 may be within a range from 25% to 75% of the overall height 298 of second sidewall 260, such as from 40% to 60%. The height 300 of second sidewall cutout 300 may be sufficiently large to allow bone preparation instrument 266 to advance to a desired location under the opposed first sidewall 258. However, the height 300 of second sidewall cutout 292 may be sufficiently small to restrict the angle at which the bone preparation instrument 266 can be advanced under the opposed sidewall (e.g., to help prevent unintended cutting in a medial direction).

In some examples, the terminal edges of first sidewall 258 and/or second side wall 260 are angled where the sidewall bounds a corresponding sidewall cutout. Angulation of the terminal edge can help when angularly aligning bone preparation instrument 266 relative to the sidewall within a corresponding cutout. For example, as shown in FIGS. 23A and 23B, first sidewall 258 can define a lower edge 302 bounding first sidewall cutout 290, and the lower edge can be angled outwardly. Similarly, second sidewall 260 can define an upper edge 304 bounding second sidewall cutout 292, and the upper edge can be angled outwardly.

With further reference to FIGS. 19 and 20, bone preparation guide 250 can be positioned over one or more bones to be prepared using the bone preparation guide. When bone preparation guide 250 includes one or more guide surfaces (e.g., slots) size smaller than a corresponding bone to be prepared, the guide surface can be aligned at different locations along the bone. In the illustrated configuration, bone preparation guide 250 is positioned such that each guide surface 254A, 256D extends over a lateral-most side of the corresponding bones to be prepared (e.g., a lateral-most side of first metatarsal 210 and medial cuneiform 222, when viewed dorsally to plantarly). When so positioned, the clinician may guide the bone preparation instrument 266 along each guide surface to cut a lateral portion of the bone directly under each guide surface. The clinician can also advance a portion of bone preparation instrument 266 beyond a medial end of bone preparation guide 250 to prepare a medial portion of the bone located beyond the medial end of the bone preparation guide.

In other examples, bone preparation guide 250 can be positioned such that each guide surface 254A, 256D extends over a medial-most side of the corresponding bones to be prepared (e.g., a medial-most side of first metatarsal 210 and medial cuneiform 222, when viewed dorsally to plantarly). When so positioned, the clinician may guide the bone preparation instrument 266 along each guide surface to cut a medial portion of the bone directly under each guide surface. The clinician can also advance a portion of bone preparation instrument 266 beyond a lateral end of bone preparation guide 250 to prepare a lateral portion of the bone located beyond the medial end of the bone preparation guide.

In still other examples, bone preparation guide 250 is positioned such that each guide surface 254A, 256D is located between a lateral-most side and a medial-most side of the corresponding bones to be prepared. When so positioned, the clinician may guide the bone preparation instrument 266 along each guide surface to cut a portion of the bone directly under each guide surface. The clinician can also advance a portion of bone preparation instrument 266 beyond a lateral end and beyond a medial end of bone preparation guide 250 to prepare a lateral portion and a medial portion, respectively, of the bone located beyond the lateral end and the medial end of the bone preparation guide.

During a surgical procedure, a clinician can insert bone preparation guide 250 through incision 100 in the skin 102 of the patient and use the bone preparation guide to prepare an end of an underlying bone, e.g., by cutting the end portion of the bone off using the bone preparation guide. In some applications, the clinician can pin bone preparation guide 250 to one or more bones to help stably position the bone preparation guide during subsequent use. Accordingly, bone preparation guide 250 can include one or fixation holes through which one or more corresponding pins can be inserted to pin the bone preparation guide to underlying bone.

With reference to FIGS. 22A and 22C, bone preparation guide 250 is illustrated as including a first arm 310 extending outwardly from body 252 of the bone preparation guide, and a second arm 312 extending outwardly from the body of the bone preparation guide. First arm 310 can define a first fixation hole 314. Second arm 312 can define a second fixation hole 316. A clinician can insert a first pin through first fixation hole 314 to pin the bone preparation guide to a first bone (e.g., the first metatarsal 210) in insert a second pin through second fixation hole 316 to pin the bone preparation guide to a second bone (e.g., medial cuneiform 222).

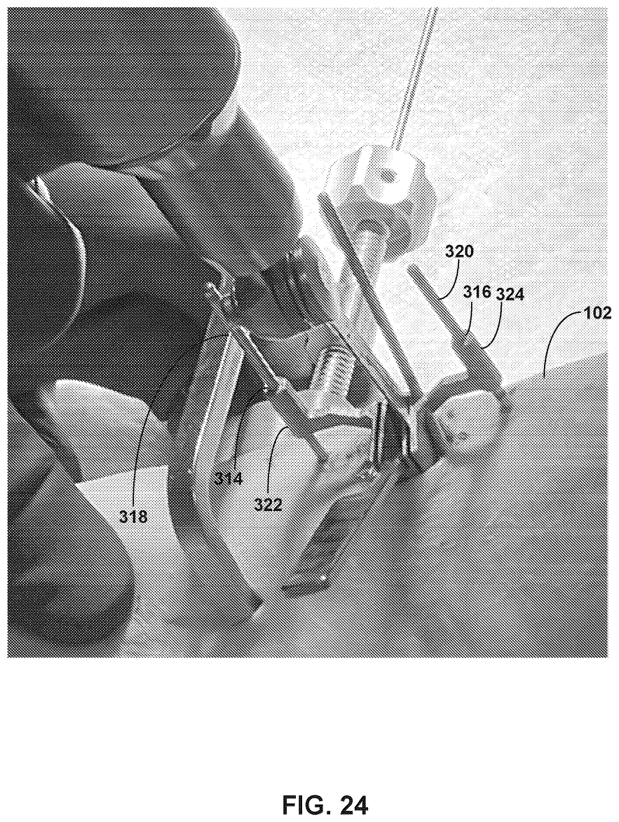

To minimize the length of incision 100 made through the skin 102 of the patient, the pins inserted through first and second fixation holes 314, 316 may be inserted percutaneously. In other words, incision 100 may not extend along the length of first metatarsal 210 and/or second metatarsal 222 a sufficient distance to expose the underlying bones over the first and second fixation holes 314, 316. Rather, the first and second fixation holes 314, 316 may be positioned over corresponding bones at a location outside of the incision (e.g., such that the patient's skin is located between the bottom surface of each respective fixation hole and the underlying bone). The clinician can advance a fixation pin through each respective fixation hole, through the skin 102 of the patient, and into the underlying bone. FIG. 24 is a perspective view of an example foot illustrating a first fixation pin 318 inserted through first fixation hole 314 and percutaneously into an underlying metatarsal and a second fixation pin 320 inserted through second fixation hole 316 and percutaneously into an underlying cuneiform.

With further reference to FIGS. 22A and 22C, first arm 310 of bone preparation guide 250 may include a first pin tower 322 defining first fixation hole 314. Additionally or alternatively, bone preparation guide 250 may include a second pin tower 324 defining second fixation hole 316. Each pin tower may include a bottom surface 326, 328 that is positioned over underlying skin and/or bone during use. In implementations, the bottom surface 326, 328 of each pin tower can contact the underlying skin when bone preparation guide 250 is pinned to the underlying bones. In other implementations, the bottom surface 326, 328 of each pin tower is elevated above the skin of the patient when the bone preparation guide is pinned to the underlying bones. Elevating the pin towers of bone preparation guide 250 above the skin during use can help prevent compression damage to the skin and/or soft tissue (e.g., tendons).

For example, bone preparation guide 250 may define a bone contacting surface 288, which may be a bottom surface of the guide that is placed in contact with one or more bones through incision 100 during use. The bottom surface 326, 328 of each pin tower may be elevated a distance 332 above the bone contacting surface 288. Distance 332 may be sufficiently large to position the bottom surface 326, 328 of each pin tower above the skin of the patient, when bone preparation guide 250 is inserted through an incision and bone contacting surface 288 is in contact with one or more bones. In some examples, distance 332 is at least 5 cm, such as at least 10 mm, at least 15 mm, at least 20 mm, at least 25 mm, at least 35 mm, or at least 45 mm. For example, distance 332 may be within a range from 15 mm to 75 mm, such as from 20 mm to 60 mm.

In addition to or in lieu of offsetting the pin towers from the surface of the skin, the arms of bone preparation guide 250 may have a length sufficient to position the pins inserted through the arms of the bone preparation guide offset from the incision 100 may to the skin of the patient. If pin towers 322, 324 are positioned too close to the ends of incision 100, respectively, the pins inserted through the pin towers may cause additional tissue damage that inhibits healing and recovery by the patient. Accordingly, pin towers 322, 324 may have a length sufficient to position corresponding pins inserted through the pin towers a distance away from the ends of the incision.

For example, first arm 310 and/or second arm 312 of bone preparation guide 250 may be sized to position the first fixation pin 318 and second fixation pin 320, respectively, at least 15 mm away from an outer sidewall surface of body 252 of bone preparation guide 250, such as at least 25 mm, at least 30 mm, at least 35 mm, at least 40 mm, at least 45 mm, or at least 50 mm.

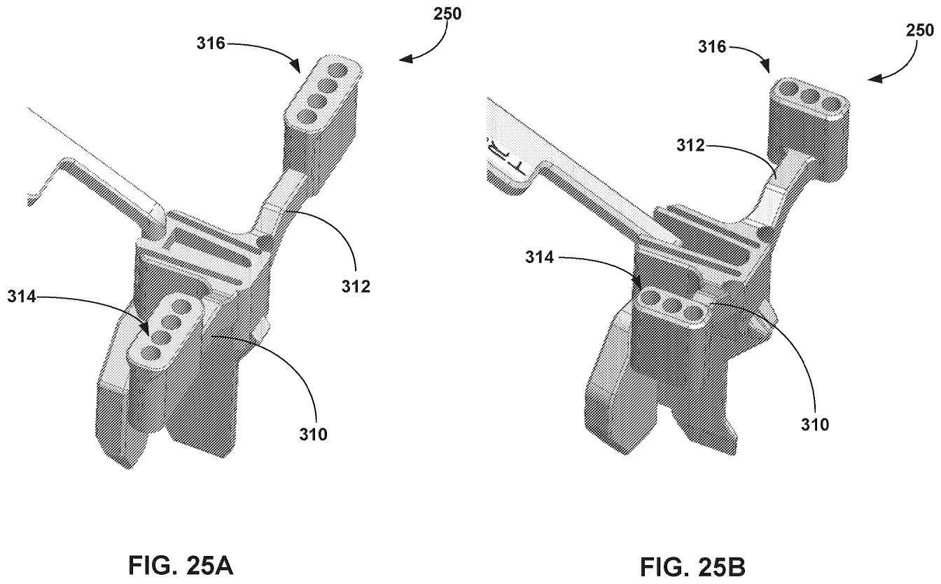

While bone preparation guide 250 in the illustrated example of FIG. 22 is shown as defining a single fixation hole (314, 316) associated with each arm on each side of the guide, in other configurations, the bone preparation guide may include multiple fixation holes defined by each arm and/or additional arms each defining one or more fixation holes. FIGS. 25A and 25B are perspective illustrations of different configurations of bone preparation guide 250 with raised of fixation holes.

FIG. 25A illustrates an example configuration of bone preparation guide 250 in which first arm 310 and second arm 312 each include a distal-proximal array of multiple fixation holes, any one or more of which can be used by the clinician to insert a fixation pin. The distal-proximal array of fixation holes may be useful to allow the clinician to insert a fixation pin at a desired location, which may be as far as possible from the end of the incision while still intersecting the desired underlying bone.

FIG. 25B illustrates an example configuration of bone preparation guide 250 in which first arm 310 and second arm 312 each include a medial-lateral array of multiple fixation holes, any one or more which can be used by the clinician to insert a fixation pin. The medial-lateral array of fixation holes may be useful to allow the clinician to insert a fixation pin at a desired location, e.g., avoiding certain soft tissue, such as to insert a pin in a target underlying bone while avoiding the EHL tendon. Although shown separately, bone preparation guide 250 may include a combination array of distal-proximal and medial-lateral spaced fixation holes.

In some examples, one or more fixation holes defined by bone preparation guide 250 are parallel to each other to allow fixation pins to be inserted through the fixation holes in parallel alignment. Bone preparation guide 250 may additionally or alternatively include one or more angled fixation apertures. For example, with reference to FIG. 18B, bone preparation guide 250 can include one or more angled fixation holes 330. As illustrated, angled fixation hole 330 extends through the body 252 of the bone preparation guide. Accordingly, when bone preparation guide 250 is inserted into an incision through the skin of the patient, a fixation pin inserted through angled fixation hole 330 may also be inserted through the incision rather than percutaneously. In other configurations, an angled fixation hole 330 may extend from body 252 of bone preparation guide 250 and be inserted percutaneously.

In use, a clinician may insert two parallel pins through fixation holes 314, 316 and may optionally insert one or more angled pins through the one or more angled fixation holes 330. This combination of parallel and angled pins may prevent bone preparation guide 250 from being removed from the underlying bones being worked upon. When the clinician has completed using the bone preparation guide, the angled pin or pins may be removed leaving the two parallel pins inserted into the underlying bones. Bone preparation guide 250 can be slid or otherwise moved up and off the parallel pins and, in some examples, a compressor (e.g., a compressor-distractor) thereafter inserted down over the pins. The compressor can then be used to apply a force to the pins to compress the prepared ends of the two facing bones together.

With further reference to FIG. 18, bone preparation guide 250 may include or be used with a spacer 340 that extends downward from the body 252. Spacer 340 may be configured to be placed into a joint (e.g., within the TMT joint). In some configurations, spacer 340 is selectively engageable with the body of the bone preparation guide and removable therefrom. The spacer can have a first portion configured to extend into a joint space and a second portion engageable with bone preparation guide body 252. For example, in the configuration shown, spacer 340 can be received within an opening 342 between sets of guide surfaces. Spacer 340 can be useful for positioning body 252 at a desired position with respect to a joint (e.g., TMT joint 230) and for properly positioning the guide with respect to bones to be cut. The distance between spacer 340 and each guide surface can define a length of tissue removal (e.g., bone or cartilage to be cut) from the underlying bone end.

When configured with spacer 340, the spacer can be inserted into TMT joint 230 after making incision 100. After inserting spacer 340 into the TMT joint, opening 342 of bone preparation guide 250 can be aligned with the spacer and the bone preparation guide installed one the spacer, thereby positioning the one or more guide surfaces defined by the bone preparation guide over one or more bone ends to be prepared. In other examples, spacer 340 may be engaged with bone preparation guide 250 prior to installation in TMT joint 230 and the spacer thereafter inserted into the joint as an assembly connected to the bone preparation guide. In still other examples, spacer 340 may be integrally and permanently attached to bone preparation guide 250.

In addition to or in lieu of using a spacer, the systems and techniques according to the disclosure may utilize a second spacer 344, which may or may not function as a fulcrum. Spacer 344 may be positionable between the proximal base of the metatarsal being moved (e.g., first metatarsal 210) and an adjacent metatarsal (e.g., second metatarsal 212). Spacer 344 can establish and/or maintain space between adjacent bones being moved, preventing and/or correcting lateral translation or base shift of the bones caused by rotation and/or pivoting.

When used, spacer 344 may be a standalone instrument, may be coupled to spacer 340 to define a bi-planar instrument, and/or may be coupled to bone preparation guide body 252 to define a combined instrument. When spacer 344 is coupled to spacer 340 and/or bone preparation guide body 252, the combination may be useful to provide a unitary structure (e.g., prior to or after being assembled) that can be positioned between two adjacent, intersecting joint spaces: a first joint space between opposed ends of a metatarsal and cuneiform and an intermetatarsal space between adjacent metatarsals.

In use, spacer 340 can be positioned at any suitable location across the joint space (e.g., in the front plane). In some examples, spacer 340 extends across the entire width of the joint space between first metatarsal 210 and medial cuneiform 222, e.g., from a medial-most end of the joint space to a lateral-most end of the joint space. In other configurations, spacer 340 extends across less than the entire width of the joint space, such as a lateral-most half or less of the joint space, or a lateral-most quarter or less of the joint space.

In some configurations, spacer 340 has a length (in the dorsal to plantar direction) sufficient such that, when the spacer body is inserted into the joint space, the spacer body projects dorsally above the joint space. In other configurations, spacer 340 may be comparatively smaller such that, when the spacer body is inserted into the joint space, the top edge of the spacer body is flush with or recessed relative to the dorsal-most surface of first metatarsal 210 and/or medial cuneiform 222 at the joint. This latter configuration can be useful to help prevent spacer 340 from visually obstructing the joint space.

Bone preparation guide body 252 may affixed to spacer 340 to define a unitary/integral instrument. The positioning of spacer 340 in the joint space can dictate the positioning of bone preparation guide body 252 coupled thereto and, correspondingly, the guiding of a bone preparation instrument facilitated by the bone preparation guide.

Spacer body 344 can be inserted between first metatarsal 210 and second metatarsal 212 (or other adjacent bones, when not performing a metatarsal realignment) concurrent with inserting spacer 340 into the TMT joint space between first metatarsal 210 and medial cuneiform 222. For example, the clinician can insert spacer 340 in the joint space between first metatarsal 210 and medial cuneiform 222 and also insert spacer 344 in the joint space between first metatarsal 210 and second metatarsal 212 at the same time. Bone preparation guide body 252 affixed to spacer 340 and/or spacer 344 can be positioned over a dorsal side of first metatarsal 210 and/or medial cuneiform 222 concurrently upon insertion of spacer 340 and/or spacer 344 into respective joint spaces.

Spacer 344 can define a length configured to be inserted into the intermetatarsal space, a thickness configured to extend between first metatarsal 210 and second metatarsal 212, and a width configured to extend in the proximal to distal direction across the foot. The thickness of spacer 344 may be substantially constant across the length of the spacer or may be tapered toward the leading end to facilitate insertion of spacer 344 into a space between adjacent metatarsals. In general, spacer 344 may have a width that extends partially within the intermetatarsal space between first metatarsal 210 and second metatarsal 212. When inserted into the intermetatarsal space, spacer 344 may extend from the base (e.g., proximal-most end) of first metatarsal 210 toward the distal-most end of the first metatarsal a distance less than half the length of the metatarsal, such as a distance less than a quarter of the length of the metatarsal, a distance less than 10% of the length of the metatarsal, or a distance less than 5% of the length of the metatarsal.

In some examples, bone preparation guide 250 includes a handle 350. Handle 350 can be operatively connected to and extend from bone preparation guide body 252. By connecting handle directly to bone preparation guide body 252, the clinician may easily manipulate the location of one or more guide surfaces defined by bone preparation guide body 252.

Spacer 344 can be operatively coupled to spacer 340. In some configurations, spacer 344 is fixedly coupled to spacer 340 to form a permanent, unmovable connection between the two spacers. In other examples, however, spacer 344 may be movably coupled to spacer 340 such that spacer 344 is rotatable relative to spacer 340. Configuring spacer 344 to be relatively rotatable to spacer 340 can be useful to allow the angle between spacer 344 and spacer 340 to be changed or manipulated by the clinician to accommodate different patient anatomies and conditions that may be encountered during a particular surgical procedure.

In some implementations, spacer 344 is rotatably coupled to spacer 340 within a bounded range of rotation. That is, spacer 344 may be mechanically coupled to spacer 340 to provide a unitary instrument but may be rotatable relative to the spacer body within a constrained or limited range of rotation. Limiting the range of rotation between spacer 344 and spacer 340 can be beneficial to allow some relative movement between spacer 344 and spacer 340 but not providing too much relative rotation such that spacer 344 becomes overly floppy or difficult for the clinician to manipulate during a surgical procedure.

In use, soft tissue may have a tendency to impinge against and/or contact the sidewalls of bone preparation guide body 252 during use, when the bone preparation guide body is inserted into incision. This may be particular true when utilizing a smaller incision 100 that limits the extent to which the skin of the patient can be retracted away from where bone preparation guide body 252 is inserted. In some configurations, bone preparation guide to may include one or more tissue deflection features to help with the positioning of soft tissue, e.g., helping to move the soft tissue away from being cut by bone preparation instrument 266.

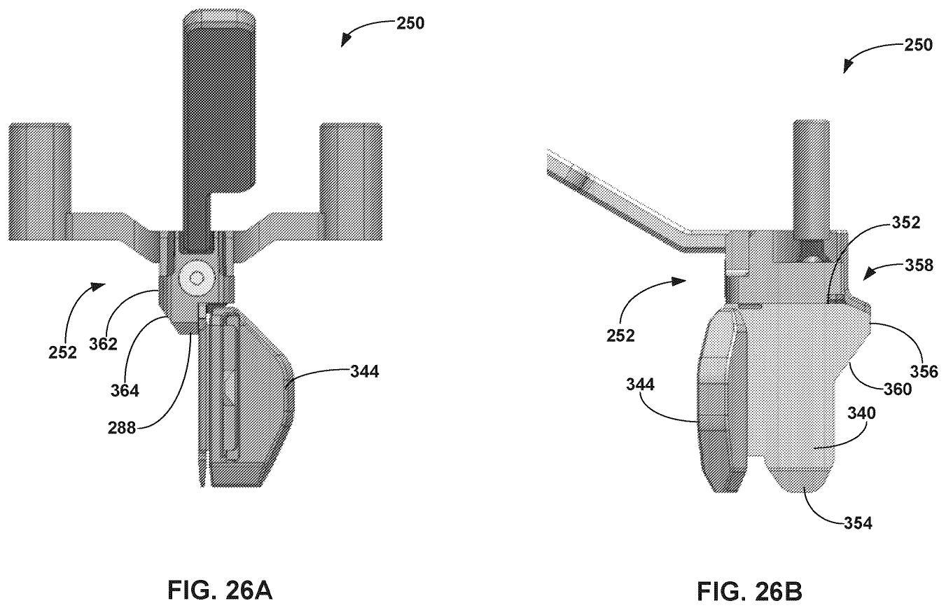

FIGS. 26A and 26B are rear and side views, respectively, of bone preparation guide 250 illustrate example tissue deflection features. As shown in this example, spacer 340 can extend from a proximal end 352 to a distal end 354 and includes an outwardly extending protrusion 356, e.g., on a proximal portion of the spacer. Protrusion 356 can define a tissue retraction space 358 into which soft tissue can be positioned. For example, during use, bone preparation guide body 252 can be inserted into incision 100 through the skin of the patient, with soft tissue being pushed dorsally along the medial edge of spacer 340 up into tissue retraction space 358. Soft tissue that may be positioned in tissue retraction space 358 can include skin and tendon(s), such as the EHL tendon. Configuring bone preparation guide 250 with such a tissue retraction space can help separate the soft tissue, such as the EHL tendon, from the cutting path traversed by a cutting instrument guided by one or more guide surfaces of the bone preparation guide. In other configurations, bone preparation guide 250 may not include a protrusion 356 extending beyond an adjacent upwardly extending sidewall (e.g., proximal sidewall). In some applications, such as when using a configuration of bone preparation guide 250 without an outwardly extending protrusion 356, the clinician may position the EHL tendon on a lateral side of the bone preparation guide 250 (e.g., pressing against a lateral wall of the guide) to offset the EHL tendon from the cutting instrument guided through the guide.

In some configurations, spacer 340 includes a ramp 360 tapering a width of the spacer across at least a portion of the length of the spacer. Ramp 360 can help facilitate insertion of spacer 340 into the TMT joint space and/or guide soft tissue being displaced during insertion of the spacer up into tissue retraction space 358. Protrusion 356 and ramp 360 are illustrated as being positioned on a medial side of bone preparation guide 250 (e.g., during use of the guide) but may additionally or alternatively be on a lateral side or another side of the guide.

In some configurations, bone preparation guide body 252 defines bottom surface 288, a sidewall 362 (e.g., a distal sidewall), and an angled surface 364 connecting the bottom surface 288 to the sidewall 362. When so configured, angled surface 364 may deflect soft tissue during insertion of bone preparation guide body 252 into incision 100 and/or during use of the bone preparation guide body. Angled surface 364 is illustrated as extending at an approximately 45° angle between bottom surface 288 and sidewall 362, although can extend at other angles.

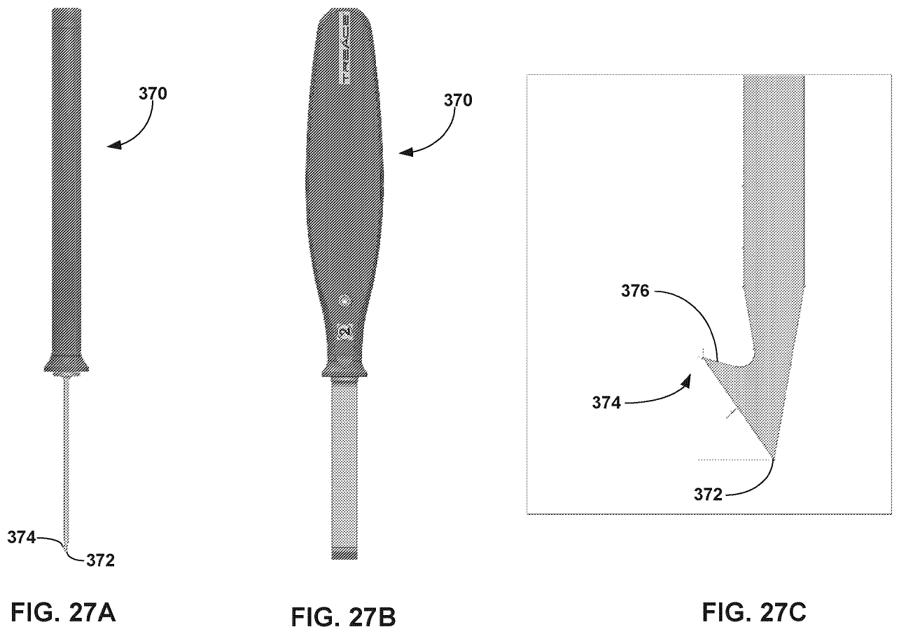

When cutting one or more bone ends, the clinician may subsequently remove a portion of cut bone from the joint space. FIGS. 27A-27C are different views of an example hooked osteotome that may be inserted into the TMT joint and used to help extract a cut bone slice from the joint. As shown, the example osteotome 370 includes a distal end 372, which may be sharpened and define a leading edge insertable into the TMT joint space. The osteotome may include a projection 374 extending outwardly and/or upwardly (e.g., plantarly) from a planar face of the osteotome. In use, projection 374 may define a ledge 376 that can be inserted under a bone slice within the TMT joint space, helping to lift the bone slide out of the joint as osteotome 370 is retracted out of the joint space.

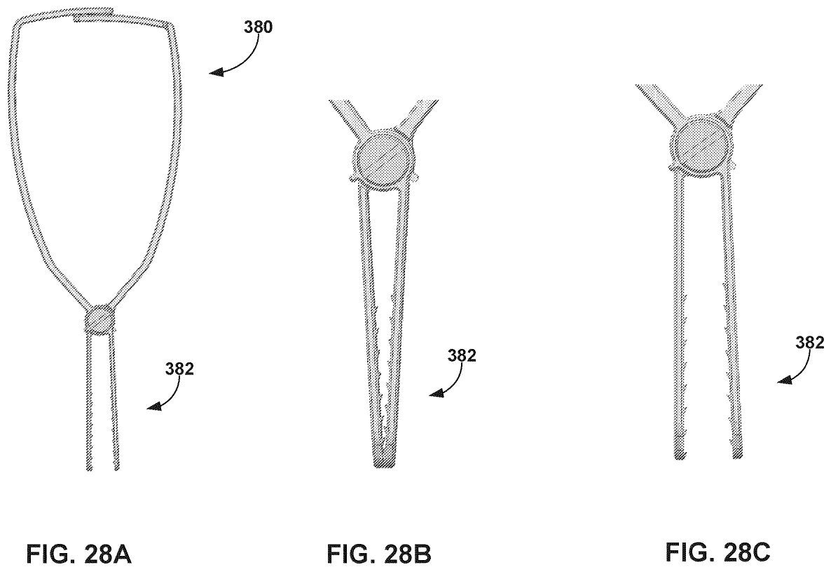

FIGS. 28A-28C are different views of an example bone slice grasping instrument 380 that may be inserted into the TMT joint and used to extract a cut bone slice from the joint. As shown, the example instrument includes a pair of opposed teeth 382 that may articulate away from each other and be closed to grasp a bone slide between the teeth. A spring or other biasing member may bias the opposed teeth 382 away from each other, with the biasing force being overcome by hand pressure by the clinician.

With further reference to FIG. 4, either before or after preparing one or both ends of first metatarsal 210 and medial cuneiform 222, the clinician may move the metatarsal in at least one plane (step 20 in FIG. 4). For example, the clinician may move metatarsal 210 in at least the transverse plane to close an intermetatarsal angle between the metatarsal and an adjacent bone (e.g., a second metatarsal) and/or a frontal plane (e.g., to reposition the sesamoid bones substantially centered under the metatarsal). In some examples, the clinician moves the bone portion in multiple planes, such as the transverse plane and/or frontal plane and/or sagittal plane. The clinician may or may not utilize a bone positioning device to facilitate movement of the bone portion.

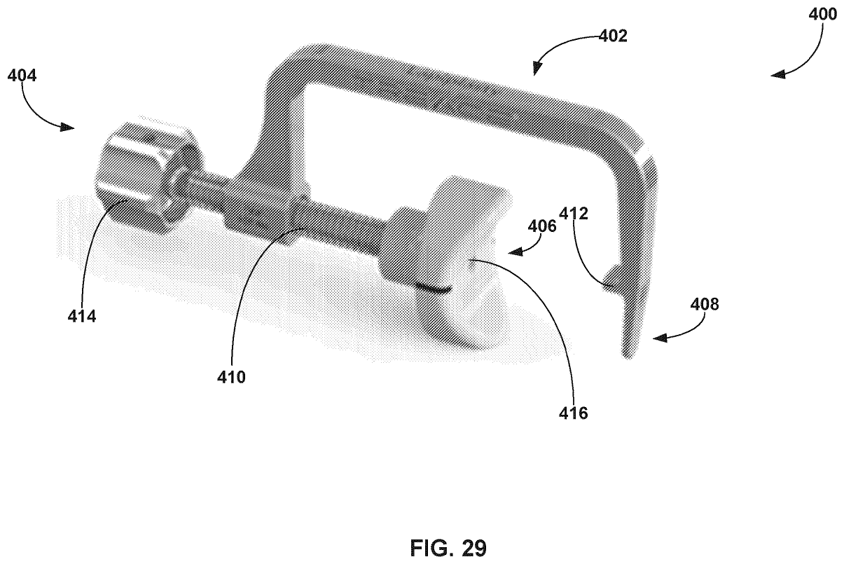

FIG. 29 shows a side perspective view of an example bone positioner 400 (also referred to as a bone positioning device) that can be used to move a metatarsal relative to an adjacent bone. In some implementations, the bone positioning device includes a metatarsal engagement member, a tip, and a mechanism to move the metatarsal engagement member and the tip relative to each other in one or more planes. For example, the mechanism may move the metatarsal engagement member and the tip towards each other (e.g. moving the metatarsal engagement member towards the tip, moving the tip towards the metatarsal engagement member, or moving both simultaneously). The bone positioning device may also include an actuator to actuate the mechanism. When the mechanism engaged, it can cause the metatarsal engaged with the metatarsal engagement member to move to correct an alignment in at least one plane with respect to a second bone in contact with the tip.

In the embodiment of FIG. 29, bone positioning device 400 includes a main body member 402, a shaft 404, a metatarsal engagement member 406 is connected to the shaft, and a tip 408 is connected to the main body member. In general, main body member 402 can be sized and shaped to clear anatomy or other instrumentation (e.g., pins and guides) while positioned on a patient. In the embodiment of FIG. 29, the main body member 402 includes a generally C-shape. In some embodiments, the main body is sized and configured to engage bones of a human foot. In addition, although bone positioning device 400 is illustrated as being formed of two components, main body member 402 and shaft 404, the guide can be fabricated from more components (e.g., three, four, or more) that are joined together to form the guide.

A shaft 404 can be movably connected to the main body member 402. In some embodiments, the shaft 404 includes threads 410 that engage with the main body member 402 such that rotation of the shaft translates the shaft with respect to the main body member. In other embodiments, the shaft can slide within the main body member and can be secured thereto at a desired location with a set screw. In yet other embodiments, the shaft can be moved with respect to the main body by a ratchet mechanism or yet other mechanism that rotates and/or linearly translates metatarsal engagement member 406 relative to tip 408. In the embodiment shown, the shaft moves along an axis that intersects the tip 508. In other embodiments, the shaft 404 and/or metatarsal engagement member 406 is offset from tip 408.

In some examples, metatarsal engagement member 406 can be configured (e.g., sized and/or shaped) to be positioned in contact with metatarsal 210 and/or an external surface of the skin 102 of the patient. For example, a force applied to and/or through metatarsal engagement member 406 can be directed through the skin to the underlying metatarsal 210 rather than directly to the metatarsal. As a result, metatarsal engagement member 406 may be positioned against the skin of the patient without making an incision to facilitate direct contact between the metatarsal engagement member 406 and metatarsal 210.

Tip 408 can be useful for contacting a bone, such as a bone different than the bone being moved by bone positioning device 400. For example, if metatarsal engagement member 406 is in contact with skin covering first metatarsal 210, the tip can be in contact with a lateral side of a different metatarsal (e.g., the second, third, fourth, or fifth metatarsal) and/or a lateral side of skin covering such metatarsal. In some examples, a small stab incision may be made between the different metatarsal and a laterally-adjacent metatarsal. For example, a stab incision may be made in an intermetatarsal space between the second metatarsal 212 and third metatarsal 214. Tip 408 of bone positioning device 400 can be inserted through the incision and the tip positioned in contact with the lateral side of the second metatarsal.

In different configurations, tip 408 may be straight or may be tapered to facilitate percutaneous insertion and contact with bone. The tip can also include a textured surface, such as serrated, roughened, cross-hatched, knurled, etc., to reduce slippage between the tip and bone. In the embodiment shown, tip 408 further includes a stop 412. Depth stop 412 can limit a depth of insertion into an intermetatarsal space (e.g., by contacting a dorsal surface of the metatarsal against which tip 408 is intended to be positioned).

As shown in FIG. 29, bone positioning device 400 can also include an actuator (e.g., a knob or a handle) 400 to actuate the mechanism, in this embodiment associated with the shaft. In the embodiment shown, the actuator can be useful for allowing a user to rotate the shaft with respect to the main body member 402. Actuator 414, shaft 404, and/or metatarsal engagement member 406 may include a cannulation 416 extending therethrough to allow the placement of a fixation wire (e.g., K-wire) through these components and into contact with or through a bone engaged with the metatarsal engagement member. For example, a fixation wire can be placed into the bone engaged with metatarsal engagement member 406 to fix the position of the metatarsal engagement member with respect to the bone. In another example, the fixation wire can be placed through the bone in contact with the metatarsal engagement member and into an adjacent bone to maintain a bone position of the bone in contact with the metatarsal engagement member and the adjacent bone.

Embodiments of the bone positioning device may include any suitable materials. In certain embodiments, the bone positioning device is fabricated at least partially from a radiolucent material such that it is relatively penetrable by X-rays and other forms of radiation, such as thermoplastics and carbon-fiber materials. Such materials are useful for not obstructing visualization of bones using an imaging device when the bone positioning device is positioned on bones.

Independent of whether the clinician utilizes a bone positioning device or the configuration of such guide, the clinician can move metatarsal 210 in one or more planes. The clinician may move the metatarsal to help correct an anatomical misalignment of the metatarsal. For example, the clinician may move the metatarsal so the metatarsal is anatomically aligned in one or more planes (e.g., two planes, three planes).

In some examples, the clinician manually moves first metatarsal 210 in addition to or moving the metatarsal through a force applied by bone positioner 400. For example, the clinician may grasp a pin inserted into the first metatarsal and/or grasp the metatarsal with a tool (e.g., forceps) and manipulate the position of the metatarsal to a desired position. In some applications, bone positioner 40 may apply a force the moves first metatarsal 210 in the transverse plane to close the IMA with an adjacent metatarsal. However, the amount of frontal plane rotation and/or sagittal plane movement achieved by bone positioner 400 through the skin of the patient may be limited. Accordingly, the clinician may manually move first metatarsal 210 (e.g., by grasping a pin inserted into the metatarsal and at least partially projecting out of the metatarsal) to rotate the metatarsal in the frontal plane and/or move the metatarsal in the sagittal plane.

In general, an anatomically aligned position means that an angle of a long axis of a first metatarsal relative to a long axis of a second metatarsal is about 10 degrees or less in the transverse plane or sagittal plane. In certain embodiments, anatomical misalignment can be corrected in both the transverse plane and the frontal plane. In the transverse plane, a normal intermetatarsal angle (“IMA”) between a first metatarsal and a second metatarsal is less than about 9 degrees. An IMA of between about 9 degrees and about 13 degrees is considered a mild misalignment of the first metatarsal and the second metatarsal. An IMA of greater than about 16 degrees is considered a severe misalignment of the first metatarsal and the second metatarsal. In some embodiments, the first metatarsal is moved to reduce the IMA from over 10 degrees to about 10 degrees or less (e.g., to an IMA of about 1-5 degrees), including to negative angles of about −5 degrees or until interference with the second metatarsal, by positioning the first metatarsal at a different angle with respect to the second metatarsal.

With respect to the frontal plane, a normal first metatarsal will be positioned such that its crista prominence is generally perpendicular to the ground and/or its sesamoid bones are generally parallel to the ground and positioned under the metatarsal. This position can be defined as a metatarsal rotation of 0 degrees. In a misaligned first metatarsal, the metatarsal is axially rotated between about 4 degrees to about 30 degrees or more. In some embodiments, methods in accordance with the invention are capable of anatomically aligning the metatarsal by reducing the metatarsal rotation from about 4 degrees or more to less than 4 degrees (e.g., to about 0 to 2 degrees) by rotating the metatarsal with respect to the medial cuneiform.

In some applications, independent of whether the clinician performs the specific bone realignment technique discussed above, the clinician may compress the end faces of the prepared bones (e.g., first metatarsal 210, medial cuneiform 222) together. For example, the technique of FIG. 4 includes optionally compressing the prepared end faces of the bone portions together prior to fixating (step 22 in FIG. 4). The clinician may compress the end faces together with hand pressure and/or using a compressing instrument physically attached to both the first bone portion and the second bone portion. For instance, the clinician may attach a compressing instrument to metatarsal 210 with one or more fixation pins and also attach the compressing instrument to medial cuneiform 222 using one or more fixation pins. In some examples, the clinician lifts bone preparation guide 250 off two or more pins that extend parallel to each other, at least one of which is inserted into metatarsal 210 and at least one of which is inserted into medial cuneiform 222. The clinician can then install a compressor back down on the parallel pins.

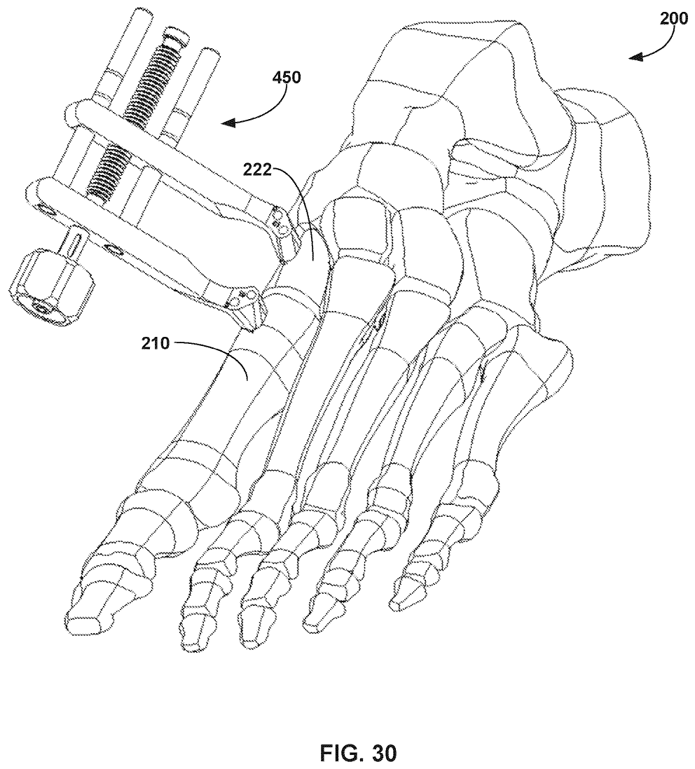

FIG. 30 is a perspective view of foot 200 illustrating an example compressing instrument 450 that may be used to compress the prepared end faces of first metatarsal 210 and medial cuneiform 222 together. Compressing instrument 450 may be attached using at least one fixation pin inserted through an arm of the compressing instrument into first metatarsal 210 and using at least one fixation pin inserted through an arm of the compressing instrument into medial cuneiform 222.

In addition to or in lieu of compressing the prepared end faces of the bones together, the technique of FIG. 4 may involve temporarily or provisionally fixating a moved position of first metatarsal 210 relative to medial cuneiform 220 prior to attaching a permanent fixation device (step 24 in FIG. 4). In some implementations, the clinician inserts one or more fixation pins through first metatarsal 210 and into an adjacent bone (e.g., second metatarsal 212, medial cuneiform 222), such as through the end faces of the first metatarsal 210 and medial cuneiform 220 in addition to or in lieu of compressing the end faces with a compressing instrument. The fixation pin may be a k-wire, olive wire (e.g., pin with region of enlarged cross-section) or other fixation pin structure. The fixation pin crossing joint 230 between first metatarsal 210 and medial cuneiform 220 can help provisionally fixate and/or compress the end faces of the two bone portions together prior to installation of permeant fixation device (and subsequent fusion of the bone faces together). When used, the one or more fixation pins can be removed from the end faces of the two bone portions and across the joint between the bone portions after installation of permanent fixation device.

Independent of whether the clinician provisionally fixates the moved position of first metatarsal 210 relative to medial cuneiform 220, the technique of FIG. 4 can involve installing one or more permanent fixation devices to first metatarsal 210 and medial cuneiform 220 across joint 230 (step 26 in FIG. 4). The one or more permanent fixation devices may hold the moved position of first metatarsal 210 relative to medial cuneiform 220, allowing the end faces of the bone portions to fuse together through a subsequent healing process.

Any one or more bone fixation devices can be used including, but not limited to, a bone screw (e.g., a compressing bone screw), a bone plate, a bone staple, an external fixator, a pin (e.g., an intramedullary implant), and/or combinations thereof. The bone fixation device may be secured on one side to metatarsal 210, bridge the TMT joint 230, and be secured on an opposite side to the proximal medial cuneiform 222. Example implants that may be used are described in U.S. provisional patent application No. 63/406,422, filed on Sep. 14, 2022; U.S. provisional patent application No. 63/444,225, filed on Feb. 8, 2023; and U.S. provisional patent application No. 63/519,039, filed on Aug. 11, 2023; the entire contents of each of which are incorporated herein by reference.

In one example, two bone implants may be placed across the TMT joint to provide bi-planar fixation. For example, a first implant (e.g., staple) may be positioned on a dorsal side of the metatarsal and medial cuneiform. A second implant may be positioned on a medial/dorsal-medial side of the metatarsal and medial cuneiform.

Various examples have been described. These and other examples are within the scope of the following claims.

1. A method of performing a minimally invasive metatarsal correction procedure, the method comprising:
positioning at least one guide surface of a bone preparation guide over a metatarsal and/or a cuneiform of a patient to be prepared, wherein the at least one guide surface has a length extending from a first end to a second end, and the length of the at least one guide surface is less than a diameter of the metatarsal and/or the cuneiform to be prepared;
guiding a bone preparation instrument along the at least one guide surface to prepare an end of the metatarsal and/or an end of the cuneiform, wherein guiding the bone preparation instrument along the at least one guide surface comprises translating the bone preparing instrument along the length of the at least one guide surface and angling the bone preparation instrument beyond one or both of the first end and the second end of the at least one guide surface to prepare the end of the metatarsal and/or the end of the cuneiform beyond one or both of the first end and the second end of the at least one guide surface;
moving the metatarsal relative to the cuneiform; and
fixating a moved position of the metatarsal.

2. The method of claim 1, wherein the length of the at least one guide surface is within a range from 10 cm to 20 cm.

3. The method of claim 1, wherein the bone preparation guide has a first sidewall defining the first end of the at least one guide surface and a second sidewall defining the second end of the at least one guide surface.

4. The method of claim 3, wherein:
the first sidewall extends from a top end to a bottom end;
the second sidewall extends from a top end to a bottom end;
the first sidewall include a first sidewall cutout extending from the bottom end toward the top end;
the second sidewall includes a second sidewall cutout extending from the top end toward the bottom end; and
angling the bone preparation instrument beyond one or both of the first end and the second end of the at least one guide surface comprises positioning the bone preparation instrument through the first sidewall cutout and the second sidewall cutout with a distal portion of the bone preparation instrument extending beyond the first sidewall.

5. The method of claim 4, wherein:
the first sidewall define a height from the top end to the bottom end and the first sidewall cutout is less than 25% of the height of the first sidewall; and
the second sidewall define a height from the top end to the bottom end and the second sidewall cutout is greater than 50% of the height of the second sidewall.

6. The method of claim 5, wherein:
the first sidewall defines a lower edge bounding the first sidewall cutout and the lower edge is angled outwardly; and
the second sidewall defines an upper edge bounding the second sidewall cutout and the upper edge is angled outwardly.

7. The method of claim 4, wherein:
positioning the at least one guide surface of the bone preparation guide over the metatarsal and/or the cuneiform to be prepared comprises positioning the second sidewall over a lateral side of the metatarsal and/or the cuneiform to be prepared; and
angling the bone preparation instrument beyond one or both of the first end and the second end of the at least one guide surface comprises angling a distal portion of the bone preparation instrument medially beyond the bottom end of the first sidewall and a proximal portion of the bone preparation instrument laterally beyond the top end of the first sidewall.

8. The method of claim 1, wherein the bone preparation guide comprise a body defining the at least one guide surface, a first arm extending outwardly from the body, and a second arm extending outwardly from the body, the first arm defining a first fixation hole and the second arm defining a second fixation hole, and further comprising:
positioning the first fixation hole over the metatarsal and inserting a first fixation pin through the first fixation hole and percutaneously into the metatarsal; and
positioning the second fixation hole over the cuneiform and inserting a second fixation pin through the second fixation hole and percutaneously into the cuneiform.

9. The method of claim 8, wherein:
positioning the first fixation hole over the metatarsal and positioning the second fixation hole over the cuneiform comprises positioning the first fixation hole and the second fixation hole offset from a skin of the patient.

10. The method of claim 8, wherein:
the body of the bone preparation guide defines a bone contacting surface;
the first arm has a first pin tower defining the first fixation hole;
the second arm has a second pin tower defining the second fixation hole; and
a bottom surface of the first pin tower and a bottom surface of the second pin tower are each elevated above the bone contacting surface of the body a distance of at least 25 mm.

11. The method of claim 8, further comprising making an incision through a skin of the patient and inserting the body of the bone preparation guide into the incision, wherein the body defines a third fixation hole, and further comprising inserting a third fixation pin through the third fixation hole of the body inserted into the incision and into an underlying bone.

12. The method of claim 1, wherein:
the at least one guide surface comprise a first guide surface and a second guide surface separated from the first guide surface;
positioning at least one guide surface of the bone preparation guide over the metatarsal and/or the cuneiform of the patient to be prepared comprises positioning the first guide surface over the metatarsal and positioning the second guide surface over the cuneiform; and
guiding the bone preparation instrument along the at least one guide surface comprises guiding the translating the bone preparing instrument along the length of the first guide surface and angling the bone preparation instrument beyond one or both of the first end and the second end of the first guide surface to prepare the end of the metatarsal and translating the bone preparing instrument along the length of the second guide surface and angling the bone preparation instrument beyond one or both of the first end and the second end of the second guide surface to prepare the end of the cuneiform.

13. The method of claim 1, further comprising, prior to positioning the at least one guide surface of the bone preparation guide over the metatarsal and/or the cuneiform:
inserting a pin percutaneously into a tarsometatarsal joint between the metatarsal and cuneiform; and
positioning an incision guide over the pin inserted percutaneously into the tarsometatarsal joint, the incision guide defining an incision guide surface for guiding an incision through a skin of the patient.

14. The method of claim 13, wherein the incision guide defines a pin receiving slot, and further comprising aligning the incision guide in a medial-to-lateral direction while the pin inserted percutaneously into the tarsometatarsal joint extends through the pin receiving slot.

15. The method of claim 1, further comprising, prior to positioning the at least one guide surface of the bone preparation guide over the metatarsal and/or the cuneiform, inserting a retraction guide into an incision through a skin of the patient and inserting a cutting instrument through the retraction guide into a tarsometatarsal joint between the metatarsal and cuneiform.

16. The method of claim 15, wherein the retraction guide includes at least one sidewall defining an enclosed opening, and inserting the retraction guide into the incision through the skin of the patient comprises positioning the skin of the patient outside of the at least one sidewall and surgically accessing the tarsometatarsal joint through the enclosed opening.

17. The method of claim 15, wherein the cutting instrument comprises an osteotome defining a depth stop sized relative to the retraction guide.

18. The method of claim 1, further comprising, prior to positioning the at least one guide surface of the bone preparation guide over the metatarsal and/or the cuneiform, inserting a bi-planar tissue release instrument at a proximal-lateral corner of the metatarsal to release soft tissue at the proximal-lateral corner of the metatarsal.

19. The method of claim 18, wherein the bi-planar tissue release instrument comprises a first cutting body joined to a second cutting body at an angle, and inserting the bi-planar tissue release instrument at the proximal-lateral corner of the metatarsal comprises inserting the first cutting body in a tarsometatarsal joint between the metatarsal and cuneiform and inserting the second cutting body between the metatarsal and an adjacent metatarsal.

20. The method of claim 19, wherein:
the first cutting body has a length extending from a proximal end to a distal end;
the second cutting body has a length extending from a proximal end to a distal end; and
the distal end of the first cutting body and the distal end of the second cutting body are each angled inwardly to define a groove therebetween.

21. The method of claim 19, wherein:
the first cutting body has a length extending from a proximal end to a distal end;
the second cutting body has a length extending from a proximal end to a distal end;
the bi-planar tissue release instrument further comprises a handle connected to the proximal end of the first cutting body and to the proximal end of the second cutting body; and
the bi-planar tissue release instrument defines an opening at the proximal end of the first cutting body and the proximal end of the second cutting body where the first cutting body is joined to the second cutting body to define the angle.

22. The method of claim 1, wherein:
positioning the at least one guide surface of the bone preparation guide over the metatarsal and/or the cuneiform further comprises inserting a spacer into a tarsometatarsal joint between the metatarsal and cuneiform;
the spacer extends from a proximal end to a distal end and includes an outwardly extending protrusion on a proximal portion of the spacer that defines a tissue retraction space; and
inserting the spacer into the tarsometatarsal joint between the metatarsal and cuneiform comprises positioning tissue of the patient in the tissue retraction space.

23. The method of claim 1, wherein the bone preparation guide comprise a body defining the at least one guide surface, the body defines a bottom surface, a sidewall, and an angled surface connecting the bottom surface to the sidewall, the angled surface deflecting tissue of the patient.

24. The method of claim 1, wherein moving the metatarsal relative to the cuneiform comprises moving the metatarsal after preparing one or both of the end of the metatarsal and the end of the cuneiform; and
further comprising, after moving the metatarsal relative to the cuneiform, compressing a prepared end of the metatarsal and a prepared end of the cuneiform together.

25. A bone preparation guide for a minimally invasive metatarsal correction procedure, the bone preparation guide comprising:
a body comprising a first sidewall, a second sidewall, and at least one guide surface having a length extending from the first sidewall to the second sidewall, the at least one guide surface being configured to guide a bone preparation instrument;
wherein one or both of the first sidewall and the second sidewall have a cutout configured to receive the bone preparation instrument extending angularly through the cutout.

26. The bone preparation guide of claim 25, wherein:
the first sidewall extends from a top end to a bottom end;
the second sidewall extends from a top end to a bottom end;
the first sidewall include a first sidewall cutout extending from the bottom end toward the top end;
the second sidewall includes a second sidewall cutout extending from the top end toward the bottom end; and
the first sidewall cutout being configured to receive a distal portion of the bone preparation instrument therein and the second sidewall cutout being configured to receive a proximal portion of the preparation instrument therein.

27. The bone preparation guide of claim 26, wherein:
the first sidewall define a height from the top end to the bottom end and the first sidewall cutout is less than 25% of the height of the first sidewall; and
the second sidewall define a height from the top end to the bottom end and the second sidewall cutout is greater than 50% of the height of the second sidewall.

28. The bone preparation guide of claim 26, wherein:
the first sidewall defines a lower edge bounding the first sidewall cutout and the lower edge is angled outwardly; and
the second sidewall defines an upper edge bounding the second sidewall cutout and the upper edge is angled outwardly.

29. The bone preparation guide of claim 25, further comprising:
a first arm extending outwardly from the body, the first arm defining a first fixation hole; and
a second arm extending outwardly from the body, the second arm defining a second fixation hole.

30. The bone preparation guide of claim 29, wherein:
the first arm positions the first fixation hole at least 10 mm from the body; and
the second positions the second fixation hole at least 10 mm from the body.

31. The bone preparation guide of claim 29, wherein:
the body of the bone preparation guide defines a bone contacting surface;
the first arm has a first pin tower defining the first fixation hole;
the second arm has a second pin tower defining the second fixation hole; and
a bottom surface of the first pin tower and a bottom surface of the second pin tower are each elevated above the bone contacting surface of the body a distance of at least 25 mm.
